(19) United States
(12) Patent Application Publication
(10) Pub. No.: US 2015/0011749 A1
(43) Pub. Date: Jan. 8, 2015

(54) USE OF CERTAIN METAL-ACCUMULATING PLANTS FOR THE PERFORMANCE OF ORGANIC CHEMISTRY REACTIONS

(71) Applicants: CENTRE NATIONAL DE LA RECHERCHE SCIENTIFIQUE, Paris (FR); UNIVERSITE MONTPELLIER 2 SCIENCES ET TECHNIQUES, Montpellier Cedex 5 (FR)

(72) Inventors: Claude Grison, Castelnau-Le-Lez (FR); Vincent Escande, Montpellier (FR)

(73) Assignees: UNIVERSITE MONTPELLIER 2 SCIENCES ET TECHNIQUES, Montpellier Cedex 5 (FR); CENTRE NATIONAL DE LA RECHERCHE SCIENTIFIQUE, Paris (FR)

(21) Appl. No.: 14/383,238

(22) PCT Filed: Mar. 5, 2013
(86) PCT No.: PCT/FR2013/050470

§ 371 Date: Sep. 5, 2014

(30) Foreign Application Priority Data
Mar. 6, 2012 (FR) 12/52045

(51) Int. Cl. B01J 23/80; C07C 41/22; C07D 333/28; C07C 67/08; C07C 231/02; B01J 21/08; C07H 3/02; C07C 67/347; C07D 223/12; C07D 487/22; B01J 31/08; B01J 21/16; C07C 45/46; C07F 7/18

(52) U.S. Cl. CPC B01J 23/80; C07C 45/46; C07C 41/22; C07D 333/28; C07C 67/08; C07C 231/02; C07F 7/188; C07H 3/02; C07C 67/347; C07D 223/12; C07D 487/22; B01J 31/08; B01J 21/16; B01J 21/08; C07C 2101/16; C07C 2102/14
USPC 536/124; 568/314; 568/656; 549/81; 560/206; 564/218; 556/467; 560/119; 540/534; 540/145; 502/159; 502/74; 502/252; 502/329; 420/513; 420/469; 420/8; 420/415; 420/402; 420/525; 75/392

(57) ABSTRACT

Metal-accumulating plants for preparing compositions including a metal catalyst derived from the plants. The composition is substantially devoid of organic matter. Also, carrying out chemical reactions with the compositions prepared from metal-accumulating plants.

The invention relates to the use of metal-accumulating plants for carrying out chemical reactions.

The biological decontamination of soils polluted with metals, metalloids, refuse and industrial and agricultural organic waste or radioisotopes is of great concern as the soil performs essential functions that largely determine the yields of food products and water quality.

Among the various pollutants, the heavy metals are some of the most harmful compounds, as they are not biodegradable and they build up in the soil. There are examples of sites in France, Belgium, Luxembourg, in the Jura, the Swiss Lower Alps or the Pyrenees, to mention just the nearest regions, as well as in regions that are more remote such as New Caledonia where there is more particularly exploitation of nickel.

Technologies for soil decontamination are difficult to develop, as it is a heterogeneous, complex and dynamic medium, which plays a key role as buffer and transformer of pollutants.

Various techniques of phytoremediation (phytoextraction, phytodegradation, phytostabilization, phytostimulation, phytotransformation, phytovolatilization and rhizofiltration) are currently in development (Terry, N. and Banuelos G., editors, Phytoremediation of contaminated soil and water, Lewis Publishers, Boca Raton, Fl. 2000).

The Centre d′Ecologie Fonctionnelle et Evolutive, (CEFE) [Centre for Functional and Evolutionary Ecology] and more particularly Doctor Escarré's team is investigating the phytostabilization technique, which consists of covering contaminated soils with plants capable of growing in the presence of heavy metals (this is called tolerance) (Frérot et al., Specific interactions between local metallicolous plants improve the phytostabilization of mine soils, Plant and Soil, 286, 53-65, 2006). Some of these plant species that are used have the particular feature of accumulating metals in large quantity in their vacuoles (they are called hyperaccumulating plants).

The team has studied two plants quite particularly; one, Thlaspi caerulescens belonging to the family Brassicaceae, has remarkable properties of tolerance and of hyperaccumulation of zinc, cadmium, and nickel. It concentrates them in the aerial parts (leaves and stems).

This plant is able to store zinc at concentrations 100 times higher than that of an ordinary plant. Moreover, it is capable of extracting and concentrating zinc and cadmium in the aerial tissues, even on soils having a low concentration of these two metals.

The other plant occurring in the mining district of Saint Laurent Le Minier, capable of accumulating large quantities of zinc, is Anthyllis vulneraria: one of the very rare leguminous plants of the flora of the temperate regions to tolerate and accumulate metals.

In addition to their unusual tolerance to Zn²⁺ and Cd²⁺, the hyperaccumulating plants are capable of extracting metals and transferring them to the aerial parts, where they accumulate. Accordingly, the roots have a very low content of heavy metals, in contrast to the non-accumulating plant species. This triple property of tolerance/accumulation/concentration in the parts that can be harvested make them a relevant tool in phytoremediation.

Moreover, the heavy metals are commonly used in organic chemistry as catalysts that are indispensable for carrying out chemical transformations that require a high activation energy. The role of the catalysts is then to lower the energy barrier.

Their manner of operation is often based on their Lewis acid properties. Zinc chloride is among those most used and is indispensable in many industrial and laboratory reactions. It is also often used in heterocyclic organic chemistry for catalysing numerous aromatic electrophilic substitutions.

It is also a catalyst of choice for carrying out hydrogenations of primary alcohols with Lucas reagent, acetalization or aldolization reactions, or cycloadditionreactions of the Diels-Alder type etc.

The catalysts are also very useful in analytical electrochemistry, electrometallurgy and liquid-solid extraction, where the fields of application are numerous and are directly involved in various areas of economic life (batteries, cells and accumulators, detectors in spectroscopy apparatus, metallurgy, welding etc.).

International application WO 2011/064462 and application WO 2011/064487 published on 3 Jun. 2011 describe and claim the invention of Professor Grison and Doctor Escarré relating to the use of a calcined plant or of a calcined plant part that has accumulated at least one metal in the M(II) form selected in particular from zinc (Zn), nickel (Ni) or copper (Cu), for preparing a composition containing at least one metal catalyst, the metal of which is one of the aforesaid metals in the M(II) form derived from said plant, said composition devoid of chlorophyll, and making it possible to carry out reactions of organic synthesis involving said catalyst.

In addition to the species mentioned above (Thlaspi caerulescens now called Noccaea caerulescens and Anthyllis vulneraria), application WO 2011/064487 describes the use of numerous other metallophyte plants hyperaccumulating heavy metals, for preparing catalysts usable in organic chemistry.

Thus, the invention described in WO 2011/064487 relates to the use of a calcined plant or of a calcined plant part that has accumulated at least one metal in the M(II) form selected in particular from zinc (Zn), nickel (Ni) or copper (Cu) as defined above, in which said plant is selected in particular from the family Brassicaceae, in particular the species of the genus Thlaspi in particular T. goesingense, T. tatrense, T. rotundifolium, T. praecox, the species of the genus Arabidopsis, in particular Arabidopsis hallerii, and of the genus Alyssum, in particular A. bertolonii, A. serpyllifolium, the Fabaceae, the Sapotaceae, in particular the species Sebertia acuminata, Planchonella oxyedra, the Convolvulaceae, in particular the species Ipomea alpina, Planchonella oxyedra, the Rubiaceae, in particular the species Psychotria douarrei, in particular P. costivenia, P. clementis, P. vanhermanii, the Cunoniaceae, in particular the Geissois, the Scrophulariaceae, in particular the species of the genus Bacopa, in particular Bacopa monnieri, the algae, in particular the red algae, in particular the rhodophyta, more particularly Rhodophyta bostrychia, the green algae or the brown algae.

Accordingly, vegetable wastes are directly utilized and transformed into “green” catalysts or into unconventional reagents.

However, the present inventors have just shown that, unexpectedly, certain plants of the genus Sedum as well as certain other plants, namely Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper have properties as metallophytes hyperaccumulating heavy metals, which make them particularly interesting for use in catalysis in organic chemistry.

The present inventors have in particular just shown that, unexpectedly, certain plants of the genus Sedum as well as a different plant, Potentilla griffithii, have properties as metallophytes hyperaccumulating heavy metals, which make them particularly interesting for use in catalysis in organic chemistry.

The plants of the genus Sedum are succulent plants that belong to the family Crassulaceae, made up of more than 400 species. They have the natural ability to develop on poor, dry soils, in an exposed situation and under difficult conditions. Their leaf system is fleshy and they are easy to grow.

Among them, two species have developed unusual properties of extracting zinc and cadmium. Sedum plumbizincicola and Sedum jinianum in particular have a remarkable capacity for extracting zinc from contaminated soils in southern and eastern China. They have real potential for phytoextraction and are described as “plumbizincicolafor”.

These properties have already been the subject of several scientific publications, among which there may be mentioned:
1—L. H. Wu, N. Li, Y. M. Luo, Phytoextraction of heavy metal contaminated soil by Sedum plumbizincicola under different agronomic strategies, in: Proc. 5th Int. Phytotech. Conf., Nanjing, China, 2008, pp. 49e50.
2—L. H. Wu, S. B. Zhou, D. Bi, X. H. Guo, W. H. Qin, H. Wang, G. J. Wang, Y. M. Luo, Sedum plumbizincicola, a new species of the Crassulaceae from Zhejiang, China. Soils 38 (2006) 632e633 (in Chinese).
3—Longhua Wu, Changyin Tan, Ling Liu, Ping Zhu, Chang Peng, Yongming Luo, Peter Christie. 2012. Cadmium bioavailability in surface soils receiving long-term applications of inorganic fertilizers and pig manures. Geoderma, 173-174: 224-230
4—Ling Liu, Longhua Wu, Na Li, Yongming Luo, Siliang Li, Zhu Li, Cunliang Han, Yugen Jiang, Peter Christie. 2011. Rhizosphere concentrations of zinc and cadmium in a metal contaminated soil after repeated phytoextraction by Sedum plumbizincicola. International Journal of Phytoremediation, 13(8): 750-764
5—Jinping Jiang, Longhua Wu, Na Li, Yongming Luo, Ling Liu, Qiguo Zhao, Lei Zhang, Peter Christie. 2010. Effects of multiple heavy metal contamination and repeated phytoextraction by Sedum plumbizincicola on soil microbial properties. European Journal of Soil Biology, 46: 18-26
6—Ling Liu, Longhua Wu, Na Li, Cunliang Han, Zhu Li, J P Jiang, Yugen Jiang, X Y Qiu, Yongming Luo, 2009. Effect of planting densities on yields and zinc and cadmium uptake by Sedum plumbizincicola. Huan Jing Ke Xue, 30 (11): 3422-67
7—Longhua Wu, Yongming Luo, Xuerong Xing and Peter Christie. 2004. EDTA-enhanced phytoremediation of heavy metal contaminated soil and associated environmental risk. Agriculture, Ecosystems & Environment, 102(3): 307-318
8—Y. T. Tang, R. L. Qiu, X. W. Zeng, R. R. Ying, F. M. Yu, and X. Y. Zhou, “Lead, zinc, cadmium hyperaccumulation and growth stimulation in Arabis paniculata Franch,” Environmental and Experimental Botany, Vol. 66, pp. 126-134, April 2009
9—R. Qiu, Y. Tang, and X. Zeng, “Method for treating soil and aquatic lead, zinc, cadmium pollution by cone south mustard,” CN1623933-A; CN1303014-C
10—H. Kubota and C. Takenaka, “Arabis gemmifera is a hyperaccumulator of Cd and Zn,” International Journal of Phytoremediation, Vol. 5, 2003 2003
11—S. L. Wang, W. B. Liao, F. Q. Yu, B. Liao, and W. S. Shu, “Hyperaccumulation of lead, zinc, and cadmium in plants growing on a lead/zinc outcrop in Yunnan Province, China,” Environmental Geology, Vol. 58, August 2009
12—W. J. Lin, T. F. Xiao, Y. Y. Wu, Z. Q. Ao, and Z. P. Ning, “Hyperaccumulation of zinc by Corydalis davidii in Zn-polluted soils,” Chemosphere, Vol. 86, pp. 837-842, February 2012
13—Z. Yanqun, L. Yuan, C. Jianjun, C. Haiyan, Q. Li, and C. Schvartz, “Hyperaccumulation of Pb, Zn and Cd in herbaceous grown on lead-zinc mining area in Yunnan, China,” Environment international, Vol. 31, pp. 755-62, 2005-July 2005
14—Y. T. Tang, R. L. Qiu, X. W. Zeng, X. H. Fang, F. M. Yu, X. Y. Zhou, et al., “Zn and Cd hyperaccumulating characteristics of Picris divaricata Vant,” International Journal of Environment and Pollution, Vol. 38, pp. 26-38, 2009
15—Q. Fang, Y. Zu, F. Zhan, Y. Li, Q. X. Fang, Y. Q. Zu, et al., “Characteristics of accumulation of Pb and Zn in Arabis alpina var. parviflora,” RDA Journal of Agro-Environment Science, Vol. 28, pp. 433-437, 2009 2009

However, the use of extracts of these plants as catalysts has never been described.

A first subject of present application is therefore the use after thermal treatment of a plant or part of a plant of the genus Sedum or of the plant Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, that has accumulated at least one metal selected in particular from zinc (Zn), copper (Cu) or iron (Fe), for preparing a composition containing at least one metal catalyst, the metal of which is one of the aforesaid metals derived from said plant, said composition substantially devoid of organic matter, for carrying out reactions of organic synthesis involving said catalyst.

A first subject of the present application is therefore the use after thermal treatment of a plant or part of a plant of the genus Sedum or of the plant Potentilla griffithii, that has accumulated at least one metal selected in particular from zinc (Zn), copper (Cu) or iron (Fe), for preparing a composition containing at least one metal catalyst, the metal of which is one of the aforesaid metals derived from said plant, said composition being substantially devoid of organic matter, for carrying out reactions of organic synthesis involving said catalyst.

A subject of the present application is therefore the use after thermal treatment of a plant or part of a plant that has accumulated at least one metal selected in particular from zinc (Zn), iron (Fe) or copper (Cu), for preparing a composition containing at least one metal catalyst, the metal of which is one of the aforesaid metals derived from said plant, said composition being substantially devoid of organic matter, for carrying out reactions of organic synthesis involving said catalyst, characterized in that the plant or plant part is of the genus Sedum or is the plant Potentilla griffithii.

A subject of the present application is also the use of a composition containing at least one metal catalyst, the metal of which is selected in particular from zinc (Zn), iron (Fe) or copper (Cu), obtained after thermal treatment of a plant or part of a plant of the genus Sedum or of the plant Potentilla griffithii that has accumulated at least one of the aforesaid metals derived from said plant, said composition being substantially devoid of organic matter, for carrying out reactions of organic synthesis involving said catalyst.

A subject of the present application is also the use of a composition containing at least one metal catalyst, the metal of which is selected in particular from zinc (Zn), iron (Fe) or copper (Cu), obtained after thermal treatment of a plant or part of a plant that has accumulated at least one of the aforesaid metals derived from said plant, said composition being substantially devoid of organic matter, for carrying out reactions of organic synthesis involving said catalyst, characterized in that the plant or plant part is of the genus Sedum or is the plant Potentilla griffithii.

More particularly, a subject of the present application is therefore the use after thermal treatment of a plant or part of a plant selected from Sedum jinianum, Sedum plumbizincicola, Sedum alfredii, Potentilla griffithii, Arabis paniculata, Arabis gemmifera and Gentiana sp. in which said at least one metal is selected from zinc (Zn), calcium (Ca), magnesium (Mg), iron (Fe), cadmium (Cd) or copper (Cu), for preparing a composition containing at least one active metal catalyst, derived from said plant, said composition having previously been filtered and/or purified on resin and/or fixed on a support, after acid treatment, for carrying out reactions of organic synthesis involving said catalyst.

The extracts of the plants which are the subject of the present invention have a different composition of the mixtures of metals relative to the extracts described in application WO 2011/064487 with in particular approximately 4 times more Zn, a greatly increased Zn/Cd, Zn/Pb ratio (knowing that the presence of cadmium and of lead is a potential drawback for these catalysts).

The presence of copper proves to be very beneficial for many syntheses. The extracts according to the invention contain very little Ni.

It also appears that the various metals present in the unpurified or partially purified mixtures have polymetallic synergy among themselves, so that these mixtures can be used in numerous reactions.

The properties of the mixtures obtained from the plants which are the subject of the present invention make them suitable for use as very efficient catalysts in a very great number of reactions, many of them not envisaged in the previous applications.

A subject of the present application is also the use as described above, in which after thermal treatment of a plant or part of a plant selected from Sedum jinianum, Sedum plumbizincicola, Sedum alfredii and Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, in which said at least one metal is selected from zinc (Zn), calcium (Ca), magnesium (Mg), iron (Fe), cadmium (Cd) or copper (Cu), for preparing a composition containing at least one active metal catalyst derived from said plant, said composition optionally having been previously filtered and/or purified on resin and/or fixed on a support, after acid treatment, for carrying out reactions of organic synthesis involving said catalyst.

A subject of the present application is also the use as described above, in which the acid treatment is carried out with hydrochloric acid, in particular gaseous HCl, 1N HCl to 12N HCl, sulphuric acid or trifluoromethanesulphonic acid.

A subject of the present application is also the use as described above after thermal treatment of a plant or part of a plant selected from Sedum jinianum, Sedum plumbizincicola, Sedum alfredii, Potentilla griffithii, Arabis paniculata, Arabis gemmifera and Gentiana sp. in which said at least one metal is selected from zinc (Zn), calcium (Ca), magnesium (Mg), iron (Fe), cadmium (Cd) or copper (Cu), for preparing a composition containing at least one active metal catalyst derived from said plant, said composition optionally having been previously filtered and/or purified on resin and/or fixed on a support, after hydration or basic treatment, for carrying out reactions of organic synthesis involving said catalyst.

A subject of the present application is also the use as described above in which the basic treatment is carried out by treating with a hydroxide, preferably sodium hydroxide or potassium hydroxide, until a pH of approximately 13 is obtained.

A subject of the present application is also the use as described above in which the composition filtered on Celite or silica is optionally subsequently purified on ion-exchange resin.

The invention also relates to a process for the preparation of a composition devoid of organic matter and comprising a metal catalyst constituted by one or more metals selected from zinc (Zn), calcium (Ca), magnesium (Mg), iron (Fe), cadmium (Cd) or copper (Cu), characterized in that it comprises the following steps:
a) dehydration of the biomass of a plant or of a plant extract preferably of the genus Sedum or of the plant Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper that has accumulated at least one metal selected from zinc (Zn), calcium (Ca), magnesium (Mg), iron (Fe), cadmium (Cd) or copper (Cu),
b) grinding of the dry biomass of a plant or of a plant extract obtained in step a),
c) thermal treatment of the ground mixture in a furnace preferably at a temperature below 500° C.
   and if desired,
d) treatment of the ash obtained in step c) with an acid preferably selected from hydrochloric acid, nitric acid, sulphuric acid, phosphoric acid or an organic acid such as trifluoromethanesulphonic acid, acetic acid, citric acid followed if desired by dehydration of the solution obtained preferably under reduced pressure so as to obtain a dry residue
   and solution obtained in step d) which, if desired, is subjected
e) to filtration preferably on Celite or on silica followed if desired by dehydration of the solution obtained preferably under reduced pressure so as to obtain a dry residue
   and/or
f) to complete or partial purification on ion-exchange resins followed if desired by dehydration of the solution obtained preferably under reduced pressure so as to obtain a dry residue
   and product in dry form obtained in step d), e) or f), which if desired
g) is mixed or treated in an acid medium with a support preferably selected from the natural or synthetic inorganic polymers or the synthetic or natural organic polymers such as silica, montmorillonite, polygalacturonic acid, chitosan, the alginates or a mixture of these products to obtain a supported catalyst.

In a preferred embodiment of the procedures for preparing the catalysts, the latter comprise steps that are common to all the preparations:
1. Dehydration of the biomass preferably in an oven at 60° C. for 1 to 2 days (the progress of dehydration is monitored by weighing until the weight has stabilized)
2. Grinding the dry leaves
3. Thermal treatment in the furnace (5 hour programme with a maximum temperature of 500° C.)

### EXAMPLE

The thermal treatment of the biomass is preferably carried out between 300 and 500° C. and ash is obtained.

In an alternative process for preparation of the ash, the step or steps of dehydration and/or grinding of the leaves may be omitted and the leaves may be calcined directly by the treatment between 300 and 500° C.

The ash may optionally be used directly if it is wished to catalyse a reaction in basic catalysis using metal oxides. The catalyst thus obtained is called CAT 1.

In all the following cases, the ash is treated with acids in solution (HCl, H₂SO₄, HNO₃, acetic acid, trifluoromethanesulphonic acid (triflic acid or TfOH) suitable for the organic syntheses envisaged.

The preferred conditions for carrying out the acid treatment are as follows:
1. Approximately 15 to 20 mL of dilute acid (1M) or concentrated acid (up to 12M) per gram of ash is introduced into the reaction mixture.
2. The reaction mixture is heated at approximately 60° C. with stirring for at least 2 hours.
3. The solution obtained is optionally filtered on Celite or silica and is optionally concentrated under reduced pressure or lyophilized.

The mineral plant extract obtained may then be used directly in unsupported catalysis or may be enriched with transition metals by partial purification on ion-exchange resins (see below) or deposited on a support for use in supported catalysis (all the other applications), depending on the requirements of organic synthesis.

### Unsupported Catalysis

For homogeneous-phase reactions, the catalysts are either used at the oxidation state existing during phytoextraction, or as co-catalysts or in reduced form (in particular Ni).

As noted above, the solution is concentrated under reduced pressure and the dry residue is then stored under a protective atmosphere (around 80° C.) in order to avoid hydration, or even hydrolysis, of the Lewis acids present. The catalyst (CAT 2) can be stored for several weeks without degradation before use.

This composition may be compared with the composition of an extract of N. caerulescens (Noccaea caerulescens, a plant also called Thlaspi caerulescens) obtained by the same process and described in international application WO 2011/064487). The ratios of elemental composition expressed as percentage by weight of the metal cations present in S. plumbizincicola relative to N. caerulescens are shown.

| | Mg | Ca | Mn | Fe | Cu | Zn | Cd | Pb |
|---|---|---|---|---|---|---|---|---|
| S. | 2.53 | 28.90 | 0.09 | 0.99 | 0.52 | 40.11 | 0.06 | 0.16 |
| plumbizincicola | | | | | | | | |
| N. caerulescens | 3.91 | 34.52 | 0.07 | 1.82 | 0.26 | 10.59 | 0.39 | 0.37 |
| SP/NC ratios | 0.6 | 1.4 | 1.3 | 0.5 | 2.0 | 3.8 | 0.1 | 0.4 |

The particularly high zinc concentration in the extract of S. plumbizincicola combined with low concentrations of Cd, Pb, Tl and As (compared to N. caerulescens) is particularly advantageous.

It should also be noted that the catalysts obtained from the plants of the genus Sedum or from the plant Potentilla griffithii contain very little nickel or are practically devoid of it.

| | Zn/Cd | Zn/Pb | Zn/Tl | Zn/As |
|---|---|---|---|---|
| S. plumbizincicola | 725 | 258 | 725 | 14698 |
| N. caerulescens | 27 | 29 | 27 | 3381 |

Other plants hyperaccumulating zinc (and optionally other metals) may be envisaged with the following zinc levels:

| | | | Dry extract after |
|---|---|---|---|
| | | | acid treatment: |
| | | dry leaves | CAT 2 |
| S. | Mean zinc level | 4% | 40% |
| plumbizincicola | (range) | (4165-45,000 mg/kg) | |
| S. jinianum | Mean zinc level | 4% | 40% |
| | (range) | (4100-41,000 mg/kg) | |
| S. alfredii | Mean zinc level | 0.5^{..}% | 5% |
| | (range) | (4134-5000 mg/kg) | |
| P. griffithii | Mean zinc level | 2% | 20% |
| | (range) | (3870-23,000 mg/kg) | |

Partial Purification on Ion-Exchange Resins if Necessary

The process for purification on ion-exchange resins is preferably carried out according to the following conditions:
1. The resin is preferably conditioned for approximately 12 hours with stirring in a solution of concentrated, for example 9M, hydrochloric acid. The flow rate in the column is adjusted to 3 mL per minute. The quantity of resin used is preferably 60 g per 1 g of product to be separated.
2. The solution obtained by hydrochloric acid treatment of the ash from the plant selected, preferably S. plumbizincicola, is fed in at the top of the column. The alkali metals and alkaline-earth metals are eluted whereas the transition metals become fixed on the resin in the form of higher chlorides. The resin may then be used as a support of transition metals for catalysis, or selective elution of the transition metals may be carried out.
3. Elution with 0.05M HCl (150 mL per gram of resin) allows the iron to be eluted.
4. Elution with 0.005M HCl and then H₂O finally allows the zinc to be eluted and the catalyst obtained corresponds to the catalyst CAT 3. A description of the process is given in the experimental section.

### Supported Catalysis

Deposition on the support may be carried out under various conditions on one and the same support or on different supports.

To use the catalysts according to the present invention in supported catalysis, mineral or organic supports may be used. Among the mineral supports there may be mentioned the aluminosilicates, such as for example the zeolites, silica SiO₂, alumina Al₂O₃, carbon, and metal oxides. It is also possible to use mixtures of the aforementioned supports as well as mining waste such as aluminosilicates laden with metal oxides.

Among the organic supports, there may be mentioned either the synthetic polymer resins and the chiral organic polymers of natural origin such as cellulose, hemicellulose, alginate, tannic acid, polygalacturonic acid, or chitosan.

Depending on the support used, it is possible to prepare Lewis acid catalysts, Lewis acid-Brønsted acid mixed catalysts, catalysts for reduction and elongation of the carbon skeleton.

The reactions that are preferably carried out by supported catalysis are the aromatic electrophilic substitution reactions, protection and deprotection of functions, rearrangements, transpositions, aldolization and related reactions, dehydration reactions, transfunctionalizations, constructions of heterocycles, multicomponent reactions, depolymerizations, redox reactions.

A catalyst supported on a zeolite such as montmorillonite K10 may be prepared for example from an unpurified plant extract, preferably of S. plumbizincicola (which corresponds to the reference CAT 4).

In a preferred embodiment, a crude plant extract, preferably of S. plumbizincicola, is introduced into an enameled crucible heated beforehand to approximately 150° C. and montmorillonite is then introduced and it is ground until a homogeneous solid is obtained. The mixture is then heated for approximately another 10 minutes before being used in organic synthesis.

The clay may be replaced with silica, and the same preparation process may be used; the catalyst is then called CAT 5.

It is also possible to prepare a Lewis acid/Brønsted acid catalyst supported on a zeolite such as montmorillonite K10 for example from an unpurified plant extract, preferably of S. plumbizincicola (which corresponds to the reference CAT 6).

In a preferred embodiment, a mixture of crude catalyst, preferably derived from Sedum plumbizincicola (Zn content: 400,000 ppm), montmorillonite K10 and 5M hydrochloric acid is heated to approximately 70° C., with stirring.

After stirring for approximately 3 hours at 70° C., the heating is increased to evaporate the medium. The solid obtained is stored in an oven (approximately 80° C.-100° C. for one to two hours) to complete its dehydration and it is ground finely in a mortar. The final Zn content of the catalyst is approximately 300,000 ppm.

A Lewis acid/Brønsted acid catalyst supported on silica may also be prepared for example from an unpurified plant extract, preferably of S. plumbizincicola (which corresponds to the reference CAT 7).

In a preferred embodiment, a mixture of catalyst preferably derived from Sedum plumbizincicola (Zn content: 400,000 ppm), silica (35-70 μm) and 5M hydrochloric acid is heated to approximately 70° C., with stirring.

The same procedure is used as previously to evaporate the medium in situ (under a hood, generally in one to two hours) and complete the dehydration of the bright yellow, sulphur-coloured solid obtained.

The final Zn content of the catalyst is approximately 300,000 ppm.

A supported catalyst may also be prepared on a mixed SiO₂/polygalacturonic acid support for example from an unpurified plant extract, preferably of S. plumbizincicola (which corresponds to the reference CAT 8).

The catalytic solution obtained after acid treatment is adjusted to pH=2 with 2M soda. The silica and the polygalacturonic acid, co-ground beforehand (the weight ratio may vary from 10/1 to 2/1), are added in solid form; the mixture is stirred for 30 minutes at ambient temperature, and then lyophilized; the solid obtained is used directly in organic synthesis.

Using the same process, the polygalacturonic acid may be replaced with chitosan, and the supported catalyst is then called CAT 9.

The Zn-hyperaccumulating plants derived from Sedum may also be used for preparing oxides and hydroxides of the transition metals. The basic properties are due to the oxygen-containing anions, and the presence of the transition metals supplies a Lewis acid character.

The metal hydroxides may thus be generated by hydration of the oxides, and then used supported or unsupported (CAT 10: unsupported, CAT 11: supported on basic alumina, CAT 12: supported on silica).

The metal hydroxides may be generated by successive treatments of the ash: acid treatment (HCl or H₂SO₄), taking up in soda at controlled pH, then operations specific to the nature of the acid (CAT 13 and CAT 14).

Basic catalysts may be prepared from accumulator plants as follows:

Various basic catalysts were prepared from the oxides resulting from thermal treatment of the biomass.
The following processes have in common that they start from metal oxides, prepared using metal-accumulating plants via the following steps:
The vegetable matter of the accumulator plants is dried in the open air or in an oven and then ground finely.
The resultant powder is subjected to a thermal treatment at 500° C. for several hours until the organic matter is completely removed. A powder consisting predominantly of oxides is obtained. These oxides are used in the following processes.

1. Preparation of a Basic Catalyst by Hydration of Oxides
A weight m of previously-prepared oxides is introduced into a flask of large volume, equipped with mechanical stirring. Water is added dropwise to the oxides, with stirring. An exothermic reaction takes place. When addition of water no longer causes a visible reaction (no swelling of the paste), the resultant mixture is homogenized, collected and left in the open air for several hours, until the paste dries naturally. During this drying, the humidity of the air completes the reaction of hydration of the oxides. Once dry, the paste is ground finely and can be used as basic catalyst. This powder must be stored under vacuum or under a nitrogen or argon atmosphere, to avoid reaction with the CO₂ in the air.

2. Preparation of Basic Catalyst by Supporting the Oxides on Silica or Basic Alumina
A weight m of previously-prepared oxides is introduced into a flask of large volume, equipped with mechanical stirring. A volume of water sufficient to completely cover the oxides is introduced very slowly, with stirring, as the reaction is exothermic. After stirring for 10 minutes, when addition of water no longer causes reaction, a weight m of silica or of basic alumina is added to the mixture. The mixture is stirred for 2 h, and then the liquid is evaporated under reduced pressure. A powder is obtained; this is ground finely and may be used as basic catalyst. This powder must be stored under vacuum or under a nitrogen or argon atmosphere, to avoid reaction with the CO₂ in the air.

3. Preparation of Basic Catalyst by Treating the Biosourced Lewis Acid Catalysts with Hydroxides
The Lewis acid catalysts derived from accumulator plant biomass, the preparation of which is described below [I have modified in this way as patents WO2011/064462 and WO2011/064487 deal with different plants even if the processes are the same in substance] are used for preparing basic catalysts by treatment with hydroxides. A weight m of biosourced Lewis acid catalyst is dissolved in water, then a concentrated solution of sodium, potassium, or calcium hydroxide, or some other metal hydroxide, is added dropwise, with stirring, and is monitored from the evolution of pH on the pH meter, until a pH of 13 is obtained. The formation of transition metal hydroxides is visible: an abundant precipitate gradually appears with increase in pH. This pH of 13 must not be exceeded, owing to the risk of the hydroxides dissolving again. The suspension is collected, centrifuged, and then dried under reduced pressure. The powder obtained is ground finely and may be used as basic catalyst. This powder must be stored under vacuum or under a nitrogen or argon atmosphere, to avoid reaction with the CO₂ in the air.

In the present application, the expressions homogeneous catalysis and unsupported catalysis must be regarded as having the same meaning. The same applies to the expressions: heterogeneous catalysis and supported catalysis.

A subject of the present application is also the use after thermal treatment of a plant or part of a plant of the genus Sedum or of the plant Potentilla griffithii that has accumulated at least one metal selected in particular from zinc (Zn), iron (Fe) and copper (Cu) for preparing a composition containing at least one metal catalyst, the metal of which is one of the aforesaid metals derived from said plant, said composition substantially devoid of organic matter for carrying out the reactions of organic synthesis of functional transformations by Lewis acid catalysis selected from the:

aromatic electrophilic substitution reactions such as Friedel-Crafts alkylating and acylating reactions and brominations, protection reactions such as chemoselective tritylations of alcohols and amines, acylations, in particular the acetylations of alcohols, phenols, thiols and amines, the silylations of alcohols, oximes, enolates, phenols, amines and anilines, the acetalizations, in particular of polyols or of sugars, the formation of imines or amines, deprotection of functions, in particular detritylation, concerted rearrangements such as the ene-reactions or cycloadditions such as the Diels-Alder reaction, the pinacol or Beckmann rearrangement, the aldolization reactions such as the Claisen-Schmidt reaction, the Mukaiyama reaction or reactions of the Knoevenagel type, dehydration or transfunctionalization reactions such as the transamination or transtritylation reactions, reactions for preparing polyheterocyclic structures such as porphyrinogens or dithienylpyrroles, multicomponent reactions such as the triazole synthesis reactions, the Hantsch and Biginelli reactions, the syntheses of piperidines, optionally substituted, of octahydroacridines, of chromenes, of pyridines and dihydropyridines, syntheses of perfume molecules such as the cyclopentenones, Jasmacyclene, campholenic aldehyde, Isobutavan, biomimetic reactions and hydride transfer reactions, depolymerization reactions, the Garcia Gonzalez reaction, reaction cascades, and redox reactions.

By “reaction cascades”, or “reactions in cascade” is meant series of consecutive intramolecular reactions involving a pericyclic reaction of the cycloaddition or electrocyclization type, and at least one other reaction of the imine formation type, reaction of the Knoevenagel type or addition. Examples are given in the experimental section.

A subject of the present application is also the use in which the composition containing at least one metal catalyst as described above and particularly a catalyst obtained according to the process described in the present application from a plant or part of a plant selected from Sedum jinianum, Sedum plumbizincicola, Sedum alfredii and Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, in which the metal is selected from zinc (Zn), calcium (Ca), magnesium (Mg), iron (Fe) with oxidation state (III), cadmium (Cd) or copper (Cu), and preferably Zn with oxidation state II is used for carrying out the reactions of organic synthesis comprising Lewis acid cocatalysis, with a catalyst of state (0) preferably obtained by reduction of a transition metal of state (II) preferably nickel obtained from plants mentioned in application WO 2011/064487. Preferably, the reaction carried out in cocatalysis is a hydrocyanation.

In the cocatalysis reactions according to the invention, the catalyst of state 0 is preferably nickel (0) obtained from nickel-hyperaccumulating plants such as preferably the plants of the genera Psychotria, Alyssum, Sebertia or Geissois. It is also possible to use other plants mentioned in application WO 2011/064487. The catalyst is used in cocatalysis with Ni(0) obtained from Ni(II) by a reduction reaction preferably with a triarylphosphite such as triphenylphosphite or tritolylphosphite to obtain a reagent of formula NiL₃ in which L represents the phosphorus-containing ligand. The cocatalyst Zn(II) may advantageously be derived from plants of the genus Sedum, obtained by the processes described in the present application.

It may be ZnCl₂ obtained by the action of HCl on the ash of plants of the genus Sedum, preferably S. plumbizincicola.

For carrying out the hydrocyanation reaction, the reagent NiL₃ is first brought into contact with HCN and then with a catalyst comprising Zn(II) preferably derived from a plant of the genus Sedum to obtain the catalyst HNiL₃CN, which is then brought into contact with the alkene on which the hydrocyanation reaction is carried out.

A subject of the present application is also the use after thermal treatment of a plant or part of a plant, different from the genus Sedum or from the plant Potentilla griffithii, that has accumulated at least one metal selected in particular from zinc (Zn), and copper (Cu) and iron (Fe), for preparing a composition containing at least one metal catalyst, the metal of which is one of the aforesaid metals derived from said plant, said composition being substantially devoid of organic matter, for carrying out reactions of organic synthesis involving said catalyst, the reactions being selected from the following reactions:

brominations, protection reactions such as chemoselective tritylations of alcohols and amines, acylations, in particular acetylations of alcohols, phenols, thiols and amines, silylations of alcohols, oximes, enolates, phenols, amines and anilines, acetalizations, in particular of polyols or of sugars, formation of imines or amines the brominations, protection reactions such as chemoselective tritylations of alcohols and amines, acylations, in particular acetylations of alcohols, phenols, thiols and amines, silylations of alcohols, oximes, enolates, phenols, amines and anilines, formation of imines or amines, deprotection of functions in particular detritylation, concerted rearrangements such as the ene-reactions or cycloadditions, the pinacol or Beckmann rearrangement, the Claisen-Schmidt reaction, the Mukaiyama reaction or reactions of the Knoevenagel type, the dehydration or transfunctionalization reactions such as the transamination or transtritylation reactions, the reactions for preparing polyheterocyclic structures such as porphyrinogens or dithienylpyrroles, multicomponent reactions such as the triazole synthesis reactions, the Hantsch reactions, the syntheses of optionally substituted piperidines, the biomimetic reactions and hydride transfer reactions, the depolymerization reactions, the Garcia Gonzalez reaction, redox reactions and reactions in cascade.

A subject of the present application is also the use after thermal treatment of a plant or part of a plant, different from the genus Sedum or from the plant Potentilla griffithii, that has accumulated at least one metal selected in particular from zinc (Zn), and copper (Cu) and iron (Fe), for preparing a composition containing at least one metal catalyst, the metal of which is one of the aforesaid metals derived from said plant, said composition being substantially devoid of organic matter, for carrying out reactions of organic synthesis involving: the deprotection of functions in particular detritylation, concerted rearrangements such as the ene-reactions or cycloadditions, the pinacol or Beckmann rearrangement, the aldolization reactions such as the Claisen-Schmidt reaction, the Mukaiyama reaction or the Knoevenagel reaction, the dehydration or transfunctionalization reactions such as the transamination or transtritylation reactions, the reactions for preparing polyheterocyclic structures such as porphyrinogens or dithienylpyrroles, the multicomponent reactions such as the triazole synthesis reactions, the Hantsch reactions, the syntheses of optionally substituted piperidines, the biomimetic reactions and hydride transfer reactions, the depolymerization reactions, the Garcia Gonzalez reaction, the redox reactions.

A subject of the present application is also the use in which the composition containing at least one metal catalyst derived from a plant, different from the genus Sedum or from the plant Potentilla griffithii, that has accumulated at least one metal selected in particular from zinc (Zn), nickel (Ni), or copper (Cu) as described above is used for carrying out the reactions of organic synthesis comprising Lewis acid cocatalysis, preferably a hydrocyanation with a catalyst of state (0) preferably obtained by reduction of a transition metal of state (II), preferably nickel.

In the following developments, by the term alkyl is meant a lower alkyl having from 1 to 8 carbon atoms, preferably from 1 to 4 carbon atoms such as methyl, ethyl, propyl, isopropyl, butyl, sec-butyl, tert-butyl, by the term aryl is meant a carbocycle such as phenyl or benzyl or a heterocyclic group such as thienyl, furyl, isothienyl, isofuryl, thiazolyl, isothiazolyl, oxazolyl, isoxazolyl, thiadiazolyl, pyridinyl or piperidinyl, and these radicals may themselves be substituted, by the term acyl is meant a group such as acetyl, propionyl, butyryl or benzoyl, by halogen is meant fluorine, chlorine, bromine or iodine.

As indicated above, the catalysts according to the invention may be used for carrying out aromatic electrophilic substitution reactions such as the Friedel-Crafts alkylating or acylating reaction. An example of such reactions is described in the following diagrams:

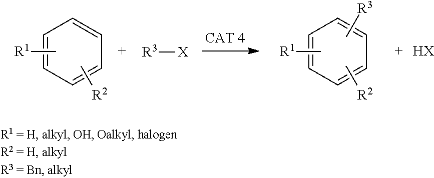

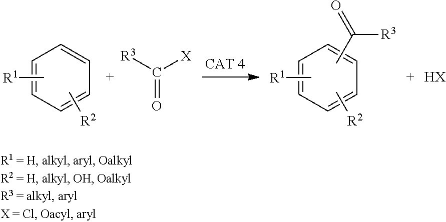

An embodiment example of a catalyst derived from Sedum plumbizincicola supported on montmorillonite K10 (CAT 4) in the preparation of a key intermediate in pharmaceutical and cosmetic chemistry is given below in the experimental section (example 1):

The catalysts according to the invention may be used for carrying out bromination reactions.

Brominated aromatic molecules are widely used by the chemical industry, equally well in the benzene, heterocyclic, and polycyclic series. These compounds are used as precursors for synthesis of molecules of economic interest, such as medicaments (examples of brominated medicinal active ingredients on the market: nicergoline, bromocriptine, brotizolam), dyes (e.g. 6,6′-dibromoindigo), flame retardants (e.g. tetrabromobisphenol A), coloured indicators (e.g. bromothymol blue).

An example of such a reaction is described in the following diagram:

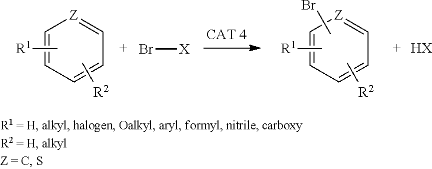

The catalysts developed from MTE-hyperaccumulating plants of the genus Sedum allow the bromination of numerous aromatic compounds by electrophilic substitution. This reaction is rapid, selective, and gives very good yields, owing to the catalyst used. The reaction can be carried out without solvent other than the bromination substrate, when the latter is liquid at the reaction temperature.

An extension of this reaction to the heterocyclic series may be represented by the following diagram:

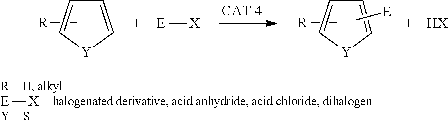

The catalysts according to the invention may be used for carrying out reactions of protection of functions, for example chemoselective tritylation of alcohols and amines according to the following diagram:

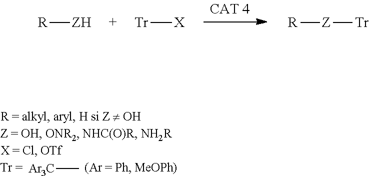

The catalysts according to the invention may be used for carrying out reactions of mild acetylations of alcohols, phenols, thiols and amines according to the following diagram:

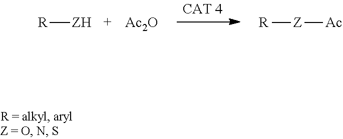

The process consisting of using the catalysts according to the invention is very advantageous owing to the ease of preparation of the supported catalyst: there is no need to handle deliquescent ZnCl₂ (the catalyst prepared is a non-sticky, finely-divided solid). Thermal activation is rapid and simple, with no loss of activity: 15 minutes at 150° C. instead of 12 hours of reflux in toluene, then 12 hours of drying at 110° C. according to Gupta et al., Ind. J. Chem. 2008, Vol. 47B, 1739-1743.

The catalysts according to the invention may be used for carrying out acetylation and silylation reactions of alcohols, phenols, amines and anilines according to the diagram:

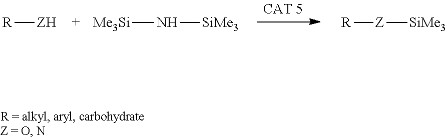

Use of the catalysts according to the invention for carrying out this step requires 10 times less catalyst than with the commercial ZnCl₂.

The catalysts according to the invention may be used for carrying out reactions of silylation of primary alcohols, of phenols or of oximes. Examples of such reactions are given in the experimental section.

The catalysts according to the invention may be used for carrying out reactions of silylation of enolates.

The originality of the results is based on the synthesis principle:

The catalysts according to the invention may be used for carrying out acetalization reactions according to the following diagram:

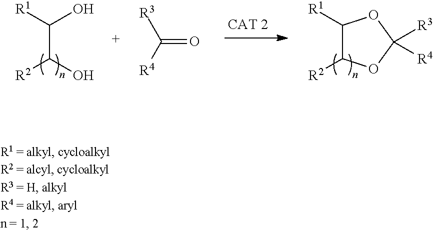

Examples of such reactions are given below in the experimental section relating to mannitol and D-glucose.

The catalysts according to the invention may be used for carrying out reactions of formation of imines according to the diagram:

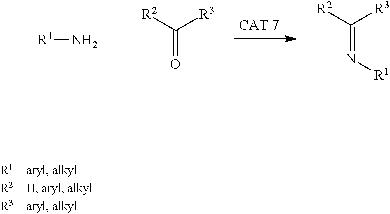

The imine formed may be isolated or used directly in a subsequent reaction of the Knoevenagel type.

The catalysts according to the invention may be used for carrying out reactions of formylation of amines according to the diagram:

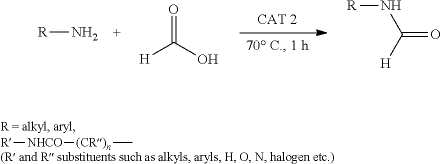

Formylation of amines is an important reaction in organic synthesis, as the formamides are used as protection for preparing peptides, as precursors of N-methyl compounds or as reagents used for the Vilsmeier-Haack formylation. The formamides are also Lewis bases used as catalysts in transformations such as hydrosilylation of carbonylated compounds.

The catalysts according to the invention may be used for carrying out reactions of deprotection of functions for example deblocking of trityl functions for example in multistep syntheses.

The catalysts according to the invention may be used for carrying out concerted rearrangements—pericyclic reactions for example the ene-reactions such as:

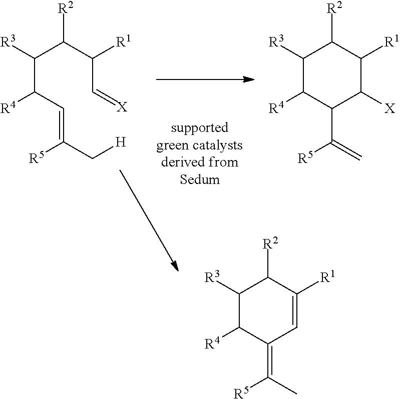

By adjusting the reaction conditions, it is possible to obtain the product resulting directly from the ene-reaction or its dehydration product.

The catalysts according to the invention may be used for carrying out cycloaddition reactions of, for example the Diels-Alder reaction.

The Diels-Alder reaction is one of the cycloadditions most exploited in organic synthesis, allowing access to complex structures such as natural products and bioactive molecules. The supported green catalysts derived from plants of the genus Sedum CAT2-9 catalyse this cycloaddition stereoselectively and lead to yields that are very high, or even quantitative, for greatly reduced reaction times. This reaction may be carried out with the supported green catalysts derived from Sedum, both in organic solvents and in the aqueous phase, which is in agreement with the principles of green chemistry. The flexibility of the process, in particular the nature of the support, which may be mineral or organic and chiral, offers numerous solutions for improving the stereochemical control of the reaction.

The results obtained show that the catalysts of the CAT 4 type lead to ratios of endo/exo products very different from 1, and much higher than in the absence of catalyst.

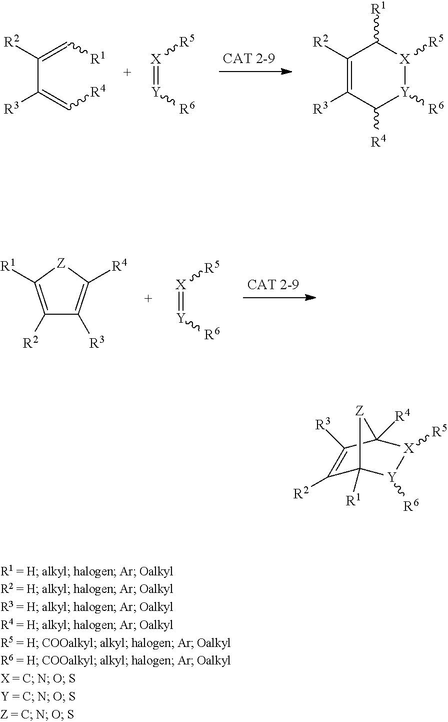

The catalysts according to the invention may be used for carrying out Diels-Alder cycloaddition reactions with asymmetric induction due to the dienophile or to the chiral support. Examples of such reactions are given below in the experimental section.

The catalysts according to the invention may be used for carrying out reactions of rearrangements, for example epoxide opening reactions according to the diagram:

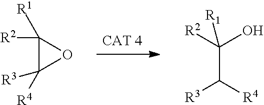

This process avoids, for the first time, the tricky preparation of magnesium dihalide in an ethereal medium. It thus becomes usable in an industrial environment.

The catalysts according to the invention may be used for carrying out reactions of pinacol rearrangement according to the following diagram:

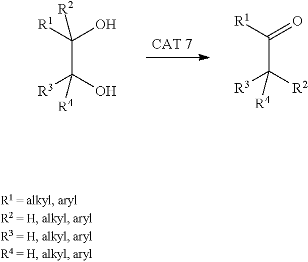

The catalysts according to the invention may be used for carrying out Beckmann rearrangement reactions according to the following diagram:

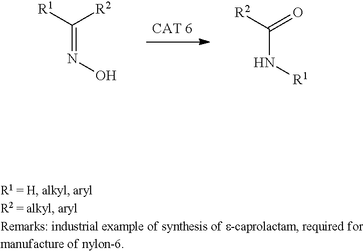

The catalysts according to the invention may be used for carrying out aldolization and related reactions:

Transformations of this type, which are very useful in organic synthesis, have found numerous examples of application with the catalysts according to the invention, where they have been shown to offer excellent performance. Numerous results are better than those described in the literature.

The catalysts according to the invention may thus be used for carrying out the Claisen-Schmidt reaction according to the following diagram:

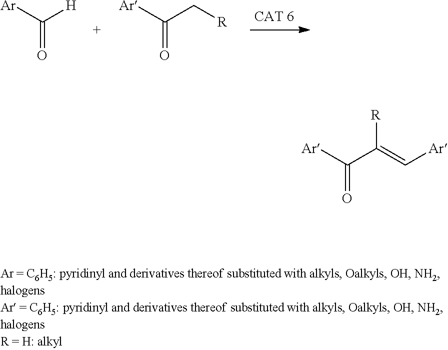

The CAT 6 catalysts derived from Sedum lead to excellent or even quantitative yields, with ethanol being used as solvent. It should be noted that the reaction mechanism involves an aldolization by acid catalysis, which is usually carried out in noxious solvents such as N,N-dimethylformamide. This aldolization is also described in the literature under basic catalysis, in ethanol, but then requires strong, aggressive bases such as NaOH and KOH, which must be removed after reaction to avoid any polluting discharges.

The catalysts according to the invention make it possible to carry out the reaction in ethanol, an environmentally friendly solvent, and without any potentially polluting and corrosive base.

The catalytic system according to the invention in particular allows the synthesis of industrially important compounds such as ionone:

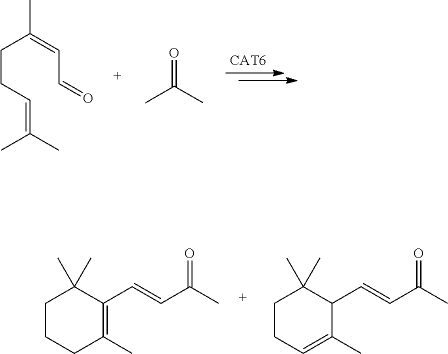

The α- and β-ionones are molecules produced on a large scale by the chemical industry, owing to their use as synthesis precursors in the pharmaceutical industry, in particular for vitamin A. The cosmetics, perfumes and flavours industry is also a large consumer of ionones, the latter being described as having violet and raspberry perfumes depending on the isomer considered. The current literature reports a synthesis route that is used very predominantly in industry, consisting of a condensation of acetone on citral, via basic catalysis (synthesis of pseudoionone) and then acid catalysis (cyclization to ionones). The bases used are aggressive (NaOH, KOH, EtONa, etc.), as too are the acids utilized in the second step of the process (sulphuric acid, phosphoric acid). Although some examples of supported catalysis of the cyclization step are reported in the literature (Díez, V.; Apesteguía, C.; Di Cosimo, J., Synthesis of Ionones by Cyclization of Pseudoionone on Solid Acid Catalysts. Catalysis Letters 2008, 123 (3), 213-219; Díez, V. K.; Marcos, B. J.; Apesteguía, C. R.; Di Cosimo, J. I., Ionone synthesis by cyclization of pseudoionone on silica-supported heteropolyacid catalysts. Applied Catalysis A: General 2009, 358 (1), 95-102), the first step of aldolization has never been carried out in supported acid catalysis, but always requires the use of bases, which have to be neutralized afterwards.

For its part, the catalytic system according to the invention allows the whole synthesis to be carried out via supported acid catalysis, based on catalysts derived from Sedum. It is therefore a “one-pot” process, and does not use any aggressive base or acid.

Two routes were explored, both leading to the synthesis of ionone by aldolization of acetone and citral in acid catalysis:
a first route (A) consists of producing the enol of acetone progressively and reacting it with citral
a second route (B) utilizes a Mukaiyama aldolization, after in situ synthesis of the silylated enol ether, in supported acid catalysis, using the catalysts derived from Sedum (for synthesis of the silylated enol ether, see the paragraph dealing with this step).

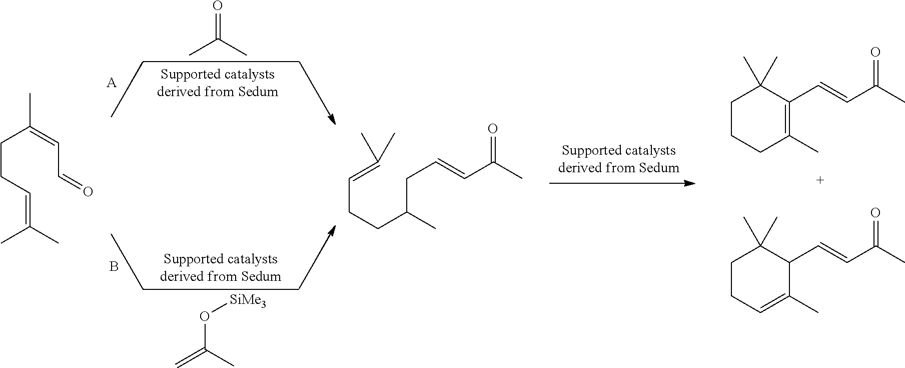

The catalysts according to the invention may thus be used for carrying out the Mukaiyama reaction according to the diagram:

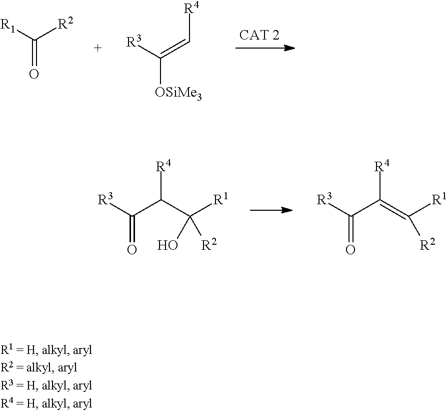

The process allows the preparation of the silylated enol ether under the conditions stated above and the Mukaiyama reaction to be linked in succession using the same catalyst derived from Sedum. This linking is unique; it has never been described in the literature.

A comparison may be made with the process described in (T. Mukaiyama and K. Narasaka, Organic Syntheses, Coll. Vol. 8, p. 323 (1993); Vol. 65, p. 6 (1987).

The catalysts according to the invention may be used for carrying out the reactions of the Knoevenagel type:

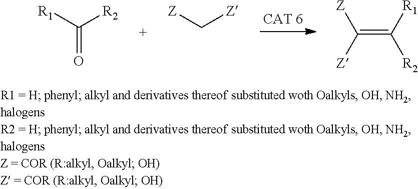

A second aldolization may take place after prolonged heating.

This reaction is described under acid catalysis but in noxious solvents such as toluene or hexane. The catalysts according to the invention derived from plants of the Sedum type make it possible to carry out this reaction in ethanol, a non-toxic solvent that can be produced from biomass, which is in agreement with the principles of sustainable chemistry. The yields obtained using ethanol as solvent are, moreover, clearly greater than those found when using other solvents, such as dichloromethane (with which the yield is only 10% for the first aldolization).

The catalysts according to the invention may be used for carrying out a reaction cascade for example a Knoevenagel reaction, hetero-Diels-Alder reaction [3+3], Diels-Alder reaction [4+2]. Such an embodiment example is described below in the experimental section.

Similarly, other reactions in cascade involving a step of the aldolization type, such as the Garcia-Gonzalez reaction, are presented in the experimental section.

The catalysts according to the invention may be used for carrying out dehydration reactions according to the diagram:

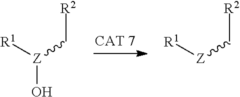

Such an embodiment example is described below in the experimental section.

The catalysts according to the invention may be used for carrying out transfunctionalization reactions.

The catalysts according to the invention may thus be used for carrying out transamination reactions according to the diagram:

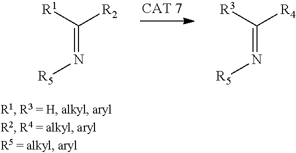

Such an embodiment example is described below in the experimental section.

The catalysts according to the invention may thus be used for carrying out transtritylation reactions according to the diagram:

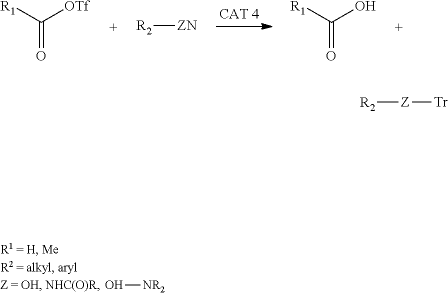

Such an embodiment example is described below in the experimental section.

The reaction is four times more rapid than that of a conventional catalysis with ZnCl₂ of commercial origin.

The catalysts according to the invention may be used for carrying out reactions for constructing simple and complex heterocycles.

The catalysts according to the invention may thus be used for carrying out reactions for preparing polyheterocyclic structures, in particular for preparing complexing pyrrole derivatives (e.g. haems of haemoglobin, chlorophyll, coenzyme B 12).

Such an embodiment example is described below in the experimental section.

The catalysts according to the invention may be used for carrying out reactions for preparing (dithenyl)pyrroles according to the diagram:

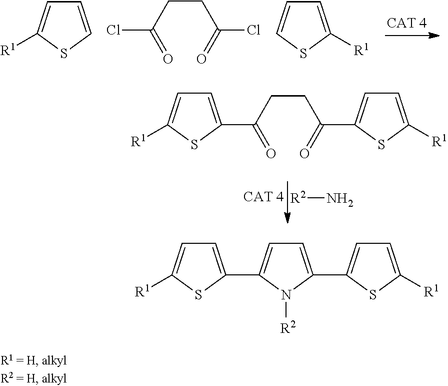

The products obtained may be used as conductive materials.

The catalysts according to the invention may be used for carrying out multicomponent reactions such as triazole synthesis according to the diagram:

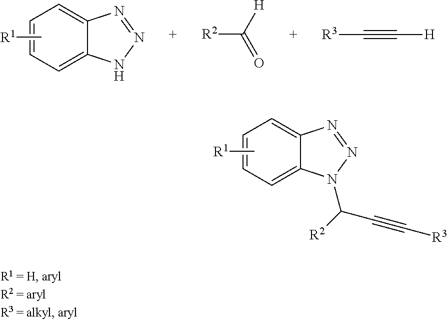

The catalysts according to the invention may be used for carrying out Hantsch and related reactions according to the diagram:

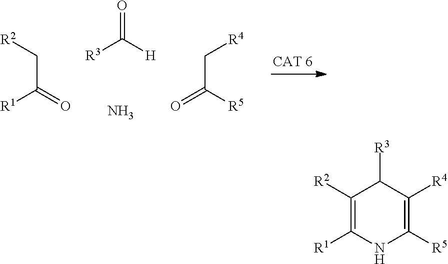

R² and R⁴ are ester groups (COOalkyl) or ketone groups (COalkyl), R¹ and R⁵ are alkyl groups, and R³ is an aryl group.

The catalysts according to the invention may be used for carrying out Biginelli reactions according to the diagram:

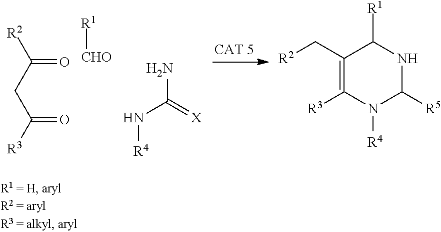

It is not necessary to purify the catalyst to obtain an efficient transformation, in contrast to the catalyst derived from Thlaspi caerulescens described in patent application WO 2011/064487.

If silica is replaced with a chiral support such as chitosan (CAT 8), commencement of asymmetric induction may be observed. This possibility is a major advantage for attaining the enantiomerically active structure of monastrol or its analogues.

The catalysts according to the invention may be used for carrying out synthesis of piperidines according to the diagram:

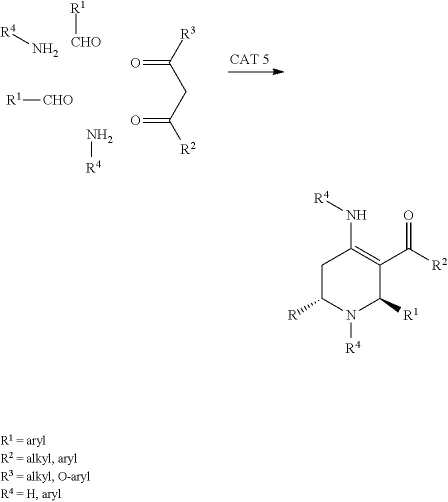

Such an embodiment example is described below in the experimental section.

The catalysts according to the invention may be used for carrying out biomimetic reductions and transfers of hydrides according to the diagram:

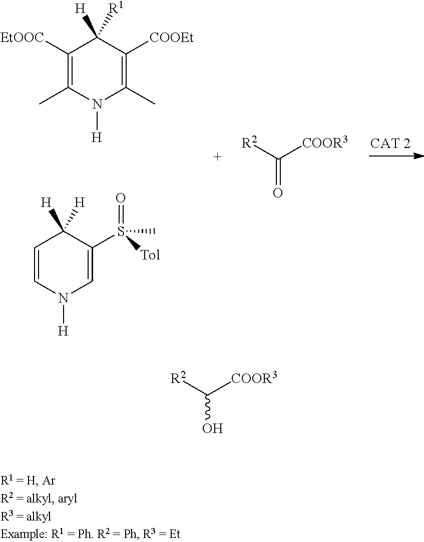

Remarks: reductions based on mimes NADH, the metal cations derived from the plant replace the enzyme

The reductions may be extended to double bonds conjugated with attracting groups such as carbonyl, carboxyl or nitro function.

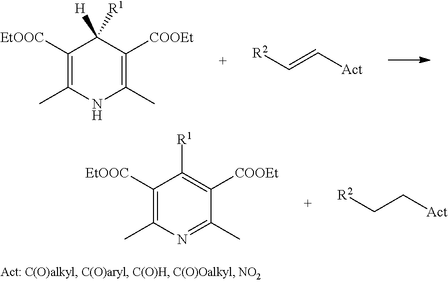

A subject of the present application is also the use of a composition containing at least one metal catalyst as described above for carrying out reactions of organic synthesis and in particular functional transformations comprising Lewis acid cocatalysis, preferably a hydrocyanation, in combination with a catalyst of state (0) preferably obtained by reduction of a transition metal of state (II), preferably nickel.

A subject of the present application is also a use in cocatalysis in which the catalyst is Ni(0) prepared by reduction of nickel(II) by the action of triphenylphosphite or tritolylphosphite on an extract of a plant that is a hyperaccumulator of Ni(II).

The Lewis acid catalysts derived from plants of the genus Sedum may play a very useful role as cocatalyst in synthesis processes involving organometallics. Among the most useful reactions, hydrocyanation of alkenes is a demonstrative example.

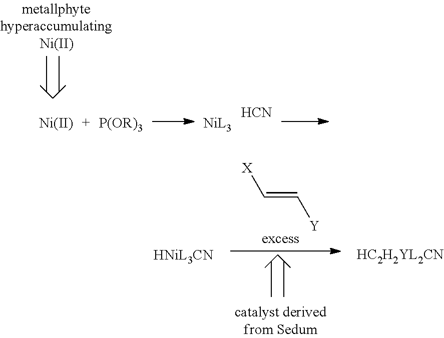

The first step of the cocatalysis reactions preferably consists of preparing an organonickel compound from metallophyte species that are hyperaccumulators of Ni(II) by the action of triphenylphosphite so as to obtain a complex of state Ni(0) of formula NiL₃.

Nickel of oxidation state zero is an efficient reagent for elongating the carbon skeleton of an aryl or of a vinyl, avoiding the magnesia or multistep routes, which are unsuitable for the current principles of green chemistry.

The catalysts of state (II), such as that derived from Psychotria douarrei, may be prepared by the process described in application WO 2011/064487.

A catalyst derived from another plant of metallophyte species that is a hyperaccumulator of Ni(II) such as Alyssum, in particular A. bertolonii, A. serpyllifolium; A. murale; Geissois pruinosa; Sebertia acuminata; Cunoniaceae, in particular the Geissois also described in WO 2011/064487 may also be used.

The present application describes for the first time the preparation of an active catalyst of metallophyte origin with two illustrative examples, the preparation of arylphosphonates and the Heck reaction. Examples of said preparation are given below in the experimental section.

A subject of the present invention is therefore the use of compositions or catalysts comprising Ni(0) obtained from extracts of metallophyte plants that are hyperaccumulators of Ni(II) for carrying out organic reactions, for example the preparation of arylphosphonates and the Heck reaction.

A subject of the present invention is therefore also the use of compositions or cocatalysts comprising, in combination, Ni(0), obtained from extracts of metallophyte plants that are hyperaccumulators of Ni(II) and a composition obtained as indicated above after thermal treatment of a plant or part of a plant of the genus Sedum or of the plant Potentilla griffithii that has accumulated at least one metal selected in particular from zinc (Zn), or copper (Cu), for carrying out organic reactions, in particular the preparation of arylphosphonates and the Heck reaction.

As indicated above, the metallophyte plants that are hyperaccumulators of Ni(II) are preferably the plants the names of which appear in international application WO 2011/064462 and application WO 2011/064487.

The reduction of Ni(II) to Ni(0) is preferably carried out with a triphenylphosphite or a tritolylphosphite (designated L hereafter).

In the second step, the hydrocyanation of alkenes is cocatalysed by metallophyte species that are hyperaccumulators of Ni(II) and of Zn(II), such as of the genus Sedum.

Formation of the final mixed species, the cocatalyst HNiL₃CN, ZnCl₂ allows alkyldinitriles to be prepared by cocatalysis with the hyperaccumulating species of the genus Sedum.

The present invention also relates to cocatalysts obtained by mixing a catalyst obtained by thermal treatment of a plant or part of a plant of the genus Sedum, preferably S. plumbizincicola or of the plant Potentilla griffithii that has accumulated at least one metal selected in particular from zinc (Zn), or copper (Cu) and a catalyst comprising Ni(0) obtained by reduction, preferably using tritolylphosphite (L), of extracts of metallophyte plants that are hyperaccumulators of Ni(II).

Among the preferred cocatalysts, there may be mentioned for example the cocatalysts of formula HNiL₃CN, ZnCl₂.

A subject of the present invention is also a process for the preparation of cocatalysts comprising a mixture of a catalyst obtained by thermal treatment of a plant or part of a plant of the genus Sedum or of the plant Potentilla griffithii that has accumulated at least one metal selected in particular from zinc (Zn), iron (Fe) or copper (Cu) and of a catalyst comprising Ni(0) obtained by reduction of an extract of metallophyte plants that are hyperaccumulators of Ni(II) for example an extract of the plant Geissois, characterized in that the extract of metallophyte plants that are hyperaccumulators of Ni(II) is subjected to the action of a triarylphosphite such as tritolylphosphite for example tri(p-tolyl) phosphite in the presence of HCN, and then the catalyst obtained by thermal treatment of a plant or part of a plant of the genus Sedum or of the plant Potentilla griffithii that has accumulated at least one metal selected in particular from zinc (Zn), iron (Fe) or copper (Cu) is added, to obtain the required cocatalyst.

The present invention also relates to the use of a cocatalyst comprising on the one hand a catalyst obtained by thermal treatment of a plant or part of a plant of the genus Sedum or of the plant Potentilla griffithii that has accumulated at least one metal selected in particular from zinc (Zn), iron (Fe) or copper (Cu) and on the other hand a catalyst comprising Ni(0) obtained by reduction of an extract obtained by thermal treatment of a plant or part of a plant of metallophyte plants that are hyperaccumulators of Ni(II) for example an extract of the plant Geissois pruinosa for preparing a composition containing at least one metal cocatalyst, said composition being substantially devoid of organic matter, for carrying out reactions of organic synthesis involving said cocatalyst.

Use according to claim 15, in which the composition containing at least one metal catalyst as described in this claim is used for carrying out the reactions of organic synthesis comprising Lewis acid cocatalysis, preferably a hydrocyanation in combination with a catalyst of state (0) preferably obtained by reduction of a transition metal of state (II), preferably nickel.

The present invention relates to the use as described in the present application in which the catalyst obtained by reduction of nickel(II) is prepared by the action of a triarylphosphite, preferably triphenylphosphite or tritolylphosphite on an extract of a plant that is a hyperaccumulator of Ni(II) which is preferably Psychotria douarrei.

A subject of the present application is also the use of a composition containing at least one metal catalyst as described above for carrying out multistep reactions of organic synthesis based exclusively on the organic catalysis of vegetable origin utilizing the Lewis acid properties of the catalysts derived from Sedum.

There may be mentioned the steps of chloromethylation, followed by cyanation and hydrochlorination/cyanation carried out with vegetable extracts according to the invention. Examples are given below in the experimental section.

There may also be mentioned the steps of protection/selective deprotection, of depolymerization/Garcia Gonzalez reaction/chemoselective protection. Examples are given below in the experimental section.

There may also be mentioned the aldolization-annelation-Diels-Alder reaction cascades. An example is given below in the experimental section.

In the description of the application stated above and hereafter, including the claims, the expression “composition containing a catalyst” or “composition containing at least one catalyst” may be replaced with “catalyst”.

A subject of the present application is thus the use after calcination of a plant or part of a plant of the genus Sedum or of the plant Potentilla griffithii that has accumulated at least one metal selected in particular from zinc (Zn), iron (Fe) and copper (Cu), for preparing a composition containing at least one metal catalyst, the metal of which is one of the aforesaid metals derived from said plant, said composition being substantially devoid of chlorophyll or of organic matter, for carrying out reactions of organic synthesis involving said catalyst.

A subject of the present application is thus the use after thermal treatment of a plant or part of a plant of the genus Sedum or of the plant Potentilla griffithii that has accumulated at least one metal selected in particular from zinc (Zn), or copper (Cu), for preparing a composition containing at least one metal catalyst, the metal of which is one of the aforesaid metals derived from said plant, said composition being substantially devoid of chlorophyll, for carrying out reactions of organic synthesis involving said catalyst.

A subject of the present application is thus the use after calcination or thermal treatment of a plant or part of a plant of the genus Sedum or of the plant Potentilla griffithii that has accumulated at least one metal in the M(II) form selected in particular from zinc (Zn), iron (Fe) or copper (Cu), for preparing a composition containing at least one metal catalyst, the metal of which is one of the aforesaid metals in the M(II) form derived from said plant, said composition being substantially devoid of chlorophyll or of organic matter, for carrying out reactions of organic synthesis involving said catalyst.

A subject of the present application is also a composition substantially devoid of or practically devoid of organic matter and in particular of chlorophyll containing at least one metal catalyst, the metal of which is selected in particular from Zn, Fe or Cu, comprising at least one of said metals in the form of chloride or sulphate, and cellulosic degradation fragments such as cellobiose and/or glucose, and/or glucose degradation products such as 5-hydroxymethylfurfural and formic acid and less than approximately 2%, in particular less than approximately 0.2% by weight of C, in particular approximately 0.14%.

In the present application, by the expression devoid of organic matter is meant that the compositions which are the subject of the invention satisfy the criteria indicated above.

In the present application, by the expression devoid of chlorophyll or devoid of organic matter is meant practically or substantially devoid of chlorophyll or of organic matter. Preferably this means that the cellulosic degradation fragments such as cellobiose and/or glucose, and/or glucose degradation products such as 5-hydroxymethylfurfural and formic acid constitute less than approximately 2%, in particular less than approximately 0.2% by weight of C, in particular approximately 0.14% of the weight of the catalyst.

A subject of the present application is also the compositions such as obtained by carrying out the various processes described above.

A subject of the present application is also the use after thermal treatment of a plant or part of a plant of the genus Sedum or of the plant Potentilla griffithii that has accumulated at least one metal selected in particular from zinc (Zn), iron (Fe) or copper (Cu), for preparing a metal catalyst, the metal of which is one of the aforesaid metals derived from said plant, said catalyst being devoid of organic matter, for carrying out reactions of organic synthesis involving said catalyst.

### Experimental Section

### Part 1

### Procedures for Preparing the Catalysts

Steps common to all the preparations:
4. Dehydration of the biomass—oven at 60° C.—1 to 2 days (the progress of dehydration is monitored by weighing until the weight has stabilized)
5. Grinding of the dry leaves
6. Thermal treatment in the furnace (5 hour programme with a maximum temperature of 500° C.)

### Example

Example of dehydration of the biomass between 300 and 500° C.
1 kg of leaves of S. plumbizincicola treated at 400° for 5 hours gives approximately 40 g of ash.
At this stage, the ash may optionally be used directly if it is wished to catalyse a reaction in basic catalysis using metal oxides (CAT 1).
In all other cases, the ash is treated with acids in solutions (for example HCl, H₂SO₄, HNO₃, H₃PO₄, trifluoromethanesulphonic acid, acetic acid) suitable for the organic syntheses envisaged.
4. 15 mL of 1-12M acid per g of ash is added to the reaction mixture.
5. The reaction mixture is heated to 60° C. with stirring for at least 2 hours.
6. The solution obtained is filtered on Celite or silica.

Preparation of Catalyst CAT 1:

217 g of dehydrated samples of Sedum plumbizincicola is calcined in a muffle furnace at 400° C. for 7 hours. 70.15 g of metal oxides is obtained.

The mineral composition in wt % of CAT 1 is given below.

| Species | Average Zn content (μg/g) |
|---|---|
| Arabis paniculata | 20,700 |
| Arabis gemmifera | 20,300 ± 1100 |
| Gentiana sp. | 19,710 ± 1602 |
| Silene viscidula | 11,155 ± 178 |
| Corydalis davidii | 9450 ± 3810 |
| Incarvillea sp. | 7000 |
| Lysmachia deltoides | 6176 ± 1640 |
| Corydalis | |
| pterygopetala | 6000 |
| Picris divaricata | 6000 |
| Arabis alpina | 5500 |
| Sonchus asper | 5000 |
| Gentiana atuntsiensis | 4528 ± 1068 |

### Examples

a/ Acid treatment between 20 and 60° C.
General diagram of the acid treatment of the ash obtained previously

MₓO_{y}+2yH⁺→xM^{2y+}+yH₂O

The preferred metal is Zn, the oxide MxOy is preferably ZnO.
The acid may be mineral (HCl) or organic; it may be diluted (for example to 1M) or concentrated (12N):

Example of acid treatment of the zinc oxide contained in the ash from S. plumbizincicola with a mineral acid, hydrochloric acid:

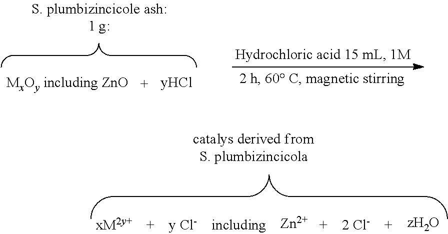

Example of acid treatment of the zinc oxide contained in the ash from S. plumbizincicola with an organic acid, trifluoromethanesulphonic acid (triflic acid or TfOH):

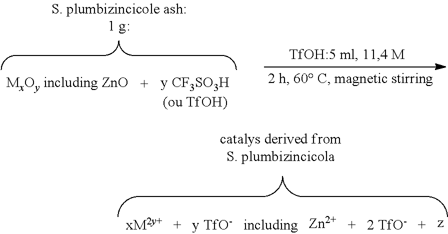

### Filtration on Celite or on Silica

The mineral plant extract obtained may then be used directly (unsupported catalysis). It may also be enriched with transition metals by partial purification on ion-exchange resins or deposited on a support (supported catalysis: all other applications), depending on the requirements of organic synthesis.
For use in unsupported catalysis (use without filtration or purification), the solution obtained above after acid treatment is concentrated under reduced pressure and the dry residue is then stored under a protective atmosphere (around 80° C.) to avoid hydration, or even hydrolysis, of the Lewis acids present. The catalyst (CAT 2) may be stored for several weeks without degradation before use.
Treatment of 1 g of ash from S. plumbizincicola treated with 20 mL of 1M HCl, for 2 hours at 60° C. followed by concentration under reduced pressure gives 1.5 g of dry extract.
The elemental composition expressed in wt % of a sample of S. plumbizincicola obtained according to the process is shown in the following table.

| | Mg | Ca | Mn | Fe | Cu | Zn | Cd | Pb |
|---|---|---|---|---|---|---|---|---|
| S. | 2.53 | 28.90 | 0.09 | 0.99 | 0.52 | 40.11 | 0.06 | 0.16 |
| plumbizincicola | | | | | | | | |

S. plumbizincicola, which is a plant that is a hyperaccumulator of zinc and other metals according to the present application has the following zinc levels:
dry leaves: mean zinc level 4%, range 4165-45,000 mg/kg
dry extract after acid treatment: (CAT 2): 40%

### Example of Use of an Amberlite® IRA 400 Resin.

1. The resin is conditioned for 12 hours with magnetic stirring in a 9M hydrochloric acid solution and then introduced into a column. The flow rate of the column is adjusted to 3 mL per minute. The quantity of resin used is 60 g per 1 g of product to be separated.
2. The solution obtained by hydrochloric acid treatment of the ash from S. plumbizincicola is introduced at the top of the column. The alkali metals and alkaline-earth metals are eluted (MgCl₂, CaCl₂, KCl) whereas the transition metals become fixed on the resin in the form of higher chlorides. The resin may then be used as support of transition metals for catalysis, or selective elution of the transition metals may be carried out.
3. Elution with 0.05M HCl (150 mL per gram of resin) allows the iron to be eluted (Zn and Pb remain on the resin in the form of higher chlorides).
4. Elution with 0.005M HCl and then H₂O finally allows the zinc to be eluted.
   The cationic composition of the dry extracts obtained from steps 3 and 4 of treatment of the resin are as follows:

| wt % | Mg | Ca | Mn | Fe | Cu | Zn | Cd | Pb |
|---|---|---|---|---|---|---|---|---|
| Step 3 | 1.8 | 13.7 | 0.3 | 6.3 | 5.0 | 7.9 | 0.1 | 0.2 |
| Step 4 | 0.7 | 1.7 | 0.2 | 0.8 | 0.1 | 43.7 | 0.1 | 0.1 |

The dry extract of cationic composition obtained in step 4 will be called CAT 3.

### Supported Catalysis

Deposition on a support may be carried out under various conditions on one and the same support or on different supports.

### Examples

Preparation of the Catalyst Supported on Montmorillonite K10: CAT 4

Co-impregnation of the catalyst—example of an unpurified extract of S. plumbizincicola supported on a zeolite, montmorillonite K10:
150 mg of crude extract of S. plumbizincicola obtained according to the procedure given below after acid treatment and filtration is introduced into an enameled crucible heated beforehand to 150° C. 200 mg of montmorillonite is then introduced and it is ground until a homogeneous solid is obtained. The mixture is then heated for another 10 minutes before being used in organic synthesis.

If the clay is replaced with silica, the catalyst is designated CAT 5 (same preparation process).

Lewis Acid/Brønsted Acid Catalyst Supported on Montmorillonite K10: CAT 6

The following are added to a 100-mL flask: 3 g of catalyst derived from Sedum plumbizincicola (Zn content: 400,000 ppm) and 1 g of montmorillonite K10. 60 mL of 5M hydrochloric acid is added and the mixture is heated to 70° C., with stiffing.
After stirring for 3 hours at 70° C., the heating is increased to evaporate the medium in situ (under a hood, generally in one to two hours). A yellow solid is obtained. The latter is stored in an oven (80° C.) overnight to complete its dehydration and then ground finely in a mortar. The final Zn content of the catalyst is approximately 300,000 ppm.

Lewis Acid/Brønsted Acid Catalyst Supported on Silica: CAT 7

The following are added to a 100-mL flask: 3 g of catalyst derived from Sedum plumbizincicola (Zn content: 400,000 ppm) and 1 g of silica (35-70 μm). 60 mL of 5M hydrochloric acid is added and the mixture is heated to 70° C., with stirring.
After stirring for 3 hours at 70° C., the heating is increased to evaporate the medium in situ (under a hood, generally in one to two hours). A bright yellow, sulphur-coloured solid is obtained. The latter is stored in an oven (100° C.) overnight to complete its dehydration and then ground finely in a mortar. The final Zn content of the catalyst is approximately 300,000 ppm.

Supported Catalyst on a Mixed SiO₂/Polygalacturonic Acid Support: CAT 8

The catalytic solution obtained after acid treatment is adjusted to pH=2 with 2M soda. The silica and the polygalacturonic acid, co-ground beforehand (weight ratio from 10/1 to 2/1), are added solid; the mixture is stirred for 30 minutes at ambient temperature, and then lyophilized; the solid obtained is used directly in organic synthesis according to the procedures described below.
If the polygalacturonic acid is replaced with chitosan, it is called CAT 9 (same process).

The basic catalysts CAT 10 to CAT 14 may be prepared as follows:
CAT 10: 5 g of metal oxides obtained from thermal treatment of Sedum is introduced into a beaker and water is added, with stirring. 30 mL of water is added. A grey suspension is obtained; stirring is maintained for 1 hour. After decanting, the pH is 10. The mixture is concentrated in a rotary evaporator, dried in an oven at 80° C. 5.041 g of a grey powder is obtained.
CAT 11: 1.684 g of metal hydroxides are co-ground with 5 g of basic alumina, then activated by 15 minutes of heating at 150° C.
CAT 12: 1.684 g of metal hydroxides are co-ground with 5 g of silica, then activated by 15 minutes of heating at 150° C.
CAT 13: 5 g of ash from Sedum is introduced into a 250-mL flask and 50 mL of 12M HCl is added with stirring. A yellowish-green solution is obtained, which is filtered on Celite. After evaporation and concentration, a bright yellow solid is collected and dried at 80° C. 2.9007 g of an ochre powder is isolated.
1.5 g of the previous solid is dissolved in 50 mL of distilled water with a few drops of HCl to promote complete dissolution. The metal hydroxides are precipitated by adding a soda solution concentrated to pH=13.3. A flocculent orange suspension is obtained. It is centrifuged at 3100 rpm. An orange-pink gel is dried under reduced pressure with slight heating. 1.069 g of solid is obtained and is stored in a desiccator under vacuum.
CAT 14: 5 g of ash from Sedum is introduced into a 250-mL flask. 100 mL of sulphuric acid is added with stirring, concentrated H₂SO₄ is added slowly to pH<1. After filtration on Celite and rinsing with distilled water, a very pale yellow solution is obtained. After concentrating in a rotary evaporator, a white powder is obtained, which is dried in an oven. 1.684 g of solid is obtained. The weight loss corresponds to removal of a large quantity of the insoluble salts: CaSO₄ and MgSO₄.
The most remarkable effect concerns the calcium salts: the level of Ca is reduced by a factor of 39 relative to the process with HCl.
This process offers a technical advantage as the salts derived from alkaline-earth cations are not of much interest in organic synthesis and do not result from the phenomenon of hyperaccumulation. This treatment is particularly well suited to biomasses with low levels of heavy metals.

The formation of metal hydroxides is identical to the process described for CAT 13.

| wt % | | | | | | |
|---|---|---|---|---|---|---|
| of cations | Mg | Ca | Fe | Cu | Zn | Al |
| CAT 1 | 2.53 | 21.26 | 26.0 | 0.51 | 38.11 | 6.91 |
| CAT 10 | 2.07 | 22.27 | 28.5 | 0.32 | 37.76 | 8.84 |
| CAT 13 | 2.36 | 20.19 | 25.9 | — | 41.09 | 8.41 |
| CAT 14 | 1.42 | 8.01 | 18.9 | — | 35.10 | 13.6 |

### Part 2

### Examples of Functional Transformations by Lewis Acid Catalysis

### Example 1

### Alkylating Friedel-Crafts Reactions

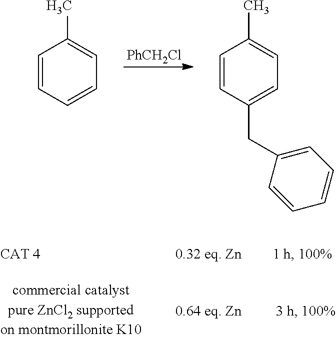

### Example 2

### Acylating Friedel-Crafts Reaction

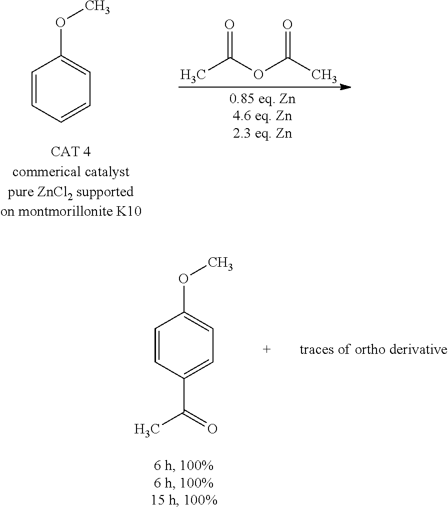

The product obtained is a key intermediate in pharmaceutical and cosmetic chemistry:

### Example 3

### Bromination

### Experimental Procedure

In a typical procedure, the liquid aromatic substrate (28 equivalents, 28 mmol) (Table 1) is introduced into a 50-mL flask equipped with a magnetic stiffing bar. The catalyst supported on montmorillonite K10 CAT 4 (150 mg of catalyst finely ground in the presence of 200 mg of K10, then dried by heating on an electric heater for 15 minutes at 150° C.) is then added to the mixture, with stirring. The reaction is carried out away from the light, in order to avoid any possible bromination by the radical route. Dibromine (1 equivalent, 1 mmol) is then added in one go, with stiffing. The reaction is completed in a few hours at ambient temperature for the compounds activated by electron-donor substituents. The deactivated compounds also react and lead to very good yields, provided the reaction mixture is heated at 60° C., under a water condenser.
In the case of compounds that are solid at the reaction temperature, these are dissolved using an organic solvent, such as dichloromethane. In a typical procedure, the solid aromatic substrate (5 equivalents, 5 mmol) (Table 1) is introduced into a 50-mL flask equipped with a magnetic stirring bar. The solid is dissolved in 3 mL of dichloromethane. The catalyst supported on montmorillonite K10 (150 mg of catalyst finely ground in the presence of 200 mg of K10, then dried by heating on an electric heater for 15 minutes at 150° C.) is then added to the mixture, with stiffing. The reaction is carried out away from the light, in order to avoid any possible bromination by the radical route. Dibromine (1 equivalent, 1 mmol) is then added in one go, with stiffing. The reaction is completed in a few hours at ambient temperature for the compounds activated by electron-donor substituents. The deactivated compounds also react and lead to very good yields, provided the reaction mixture is heated at 60° C., under a water condenser.

TABLE 1

| Substrate | Product | Time | Temperature | Yield |
|---|---|---|---|---|
| 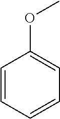 | 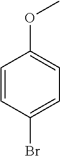 | 6 h | 25° C. | 100% |
| 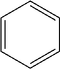 | 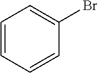 | 3 h | 60° C. | 100% |
| 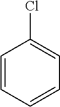 | 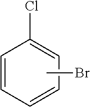 | 6 h | 60° C. | 100% |
| 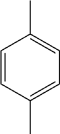 | 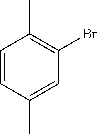 | 3 h | 60° C. | 100% |
| 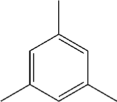 | 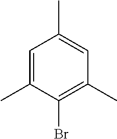 | 3 h | 60° C. | 100% |
| 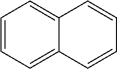 | 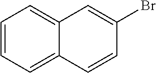 [SMILES: Brc1ccc2ccccc2c1] | 3 h | 40° C. | 100% |
| 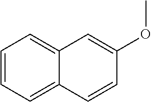 | 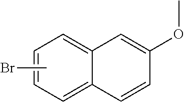 | 17 h | 25° C. | 83% |
| 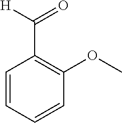 | 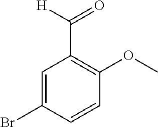 | 3 h | 40° C. | 100 % |
| 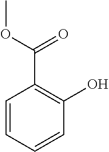 | 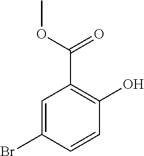 | 3 h | 60° C. | 100 % |

### Example 4

### Extension of Bromination to the Heterocyclic (Thiophene) Series

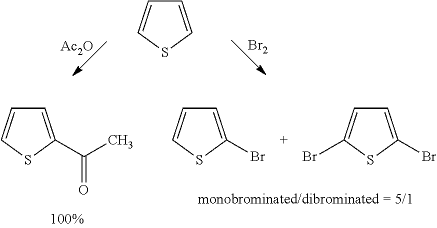

| Substrate | Product | Time | Temperature | Yield |
|---|---|---|---|---|
| 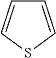 | 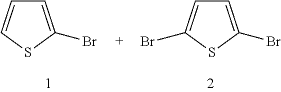 | 1 h 30 | 25° C. | 45% (1); 25% (2) (1)/(2) = 1.8 |
| 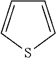 | 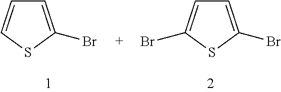 | 2 h | 0° C. | 36% (1); 8% (2) (1)/(2) = 4.6 |

In the second example above, the reaction conditions are as follows: thiophene (1 mmol)+dibromine (1 mmol) diluted in 5 mL of dichloromethane, stirred in an ice bath, with dropwise addition of dibromine

### Example 5

### Protection of Functions: Chemoselective Tritylations of Alcohols and Amines

### Examples

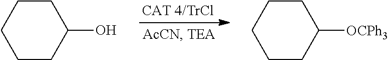

In a flask taken out of the oven, 5 mL of acetonitrile is added and then, with magnetic stirring, 1 mmol of cyclohexanol (108 mg) and 1 mmol of trityl chloride (278.8 mg). The supported catalyst is prepared by co-grinding by the process described above. 94 mg of crude extract of S. plumbizincicola (0.58 mmol Zn) is used per 170 mg of montmorillonite K10 (CAT 4). The catalyst is added after activation to the reaction mixture obtained in 1. After stirring for 5 minutes, 1 mmol (135 μL) of triethylamine in solution in 2 mL of acetonitrile is added. Stirring is maintained for 1 hour. After one hour, the reaction is stopped by adding 10 mL of 5% citric acid buffer. After stirring for another 5 minutes, the catalyst is separated by filtration, and the reaction mixture is concentrated under reduced pressure. It is taken up in dichloromethane and the organic phase is washed with water. After drying and concentrating the organic phase, the product obtained is analysed by infrared spectrometry and GC MS. Menthol is used as internal reference and allows confirmation that the reaction is quantitative.

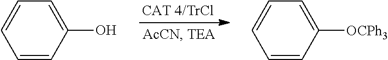

The process is identical to the previous one. The reaction is monitored by IR (shift of the carbonyl vibrator from 1684 to 1728 cm⁻¹). The end product is characterized by its melting point (Mp: 168° C.).

### Example 6

### Mild Acetylations of Alcohols, Phenols, Thiols and Amines

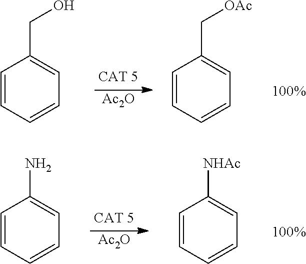

In a typical procedure, the nucleophilic substrate (1 mmol) is introduced into a 10-mL flask equipped with a magnetic stirring bar. 1.2 mmol of acetic anhydride diluted in 10 mL of acetonitrile is added. The silica-supported catalyst CAT 5 (94 mg of catalyst finely ground in the presence of 170 mg of SiO₂, then dried by heating on an electric heater for 15 minutes at 150° C.) is then added to the mixture, with stirring. The reaction is complete in 3 hours at 80° C. The reaction mixture is filtered and the catalyst is isolated and dried for a subsequent reaction. The filtrate is diluted in an organic solvent such as dichloromethane, washed with a dilute solution of sodium hydrogen carbonate, dried and concentrated. IR (vibrator C═O) and then GC MS confirm the quantitative formation and purity of the acetylation products.

### Example 7

### Silylations of Alcohols, Phenols, Amines and Anilines

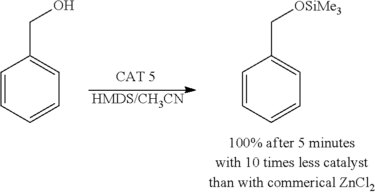

### Example 7a

### Silylation of Primary Alcohols: Example of Cyclohexanol and of Benzyl Alcohol

The nucleophilic substrate (1 mmol) is introduced into a 10-mL flask equipped with a magnetized bar for magnetic stirring and a CaCl₂ trap. 0.75 mmol of hexamethyldisilazane (HMDS) diluted in 2 mL of acetonitrile is added. The silica-supported catalyst derived from Sedum CAT 5 (9.4 mg of catalyst is finely ground, i.e. equivalent to 0.024 mmol of ZnCl₂, in the presence of 17 mg of SiO₂, then dried by heating on an electric heater for 15 minutes at 150° C.) is then added to the mixture, with stirring. The reaction is complete in 15 minutes at ambient temperature. The reaction mixture is filtered, then evaporated and the catalyst is isolated and then dried for a subsequent reaction.
IR (from absence of —OH vibration bands) and then GC MS and ¹H NMR confirm the quantitative formation and purity of the silylation product.

### Example 7b

### Silylation of Phenols: Example of Phenol

The procedure is the same as for the primary alcohols. The reaction is also complete in 15 minutes at ambient temperature. Moreover, IR (from absence of —OH vibration bands) and then GC MS and ¹H NMR confirm the quantitative formation and purity of the silylation product.

### Example 7c

### Silylation of Oximes: Example of Benzaldehyde Oxime

The procedure is the same as for the primary alcohols. The reaction is also complete in 20 minutes at ambient temperature. Similarly, IR (from absence of —OH vibration bands) and then GC MS and ¹H NMR confirm the quantitative formation and purity of the silylation product.
The process is very advantageous owing to the ease of preparation of the supported catalyst: there is no need to handle deliquescent ZnCl₂ (the catalyst prepared, CAT 5, is a non-sticky, finely-divided solid). Thermal activation is rapid and simple with no loss of activity: 15 minutes at 150° C. instead of 3 hours at 150° C. and then 20 minutes of grinding at 30° C. and 2 hours of drying at 80° C. under vacuum according to Upadhyaya D. J. and Samant S. D., 2008. Moreover, the yields are just as satisfactory as those of Shaterion H. R. et al., 2009 using this catalyst the preparation of which is simpler, less expensive in energy terms and more rapid.
It is also possible to carry out partial or complete silylation of a carbohydrate such as D-glucose diethyl mercaptan, which is used as a model (Part 4, ex. 2 i).

### Example 8

### Acetalization

### Example 8a

### Acetalization of Mannitol

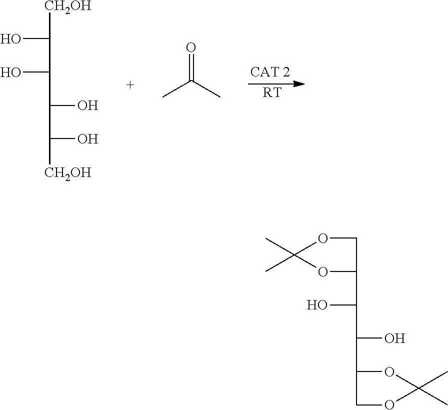

500 mg of catalyst CAT 2 derived from plants of the genus Sedum and 2690 mg (46 mmol, 20 equiv) of anhydrous acetone are introduced into a four-necked flask, under a dinitrogen stream. After stirring, the medium is decanted and the supernatant is siphoned into a second four-necked flask containing 425 mg (2.3 mmol, 1 equiv) of D-mannitol. The solution is stirred vigorously for 2.5 hours and then filtered on a frit before being added in one go to a mixture of 850 mg of K₂CO₃, 0.850 mL of water and 0.335 mL of ether and then stirred vigorously for 40 minutes. The solution is decanted, and the precipitate is washed with an acetone/ether 1/1 mixture. The organic phases are dried over K₂CO₃, filtered and then evaporated. The viscous solid obtained is dissolved hot in 5 mL of toluene and then recrystallized at low temperature after addition of an equivalent volume of hexane.

### Example 8b

### Acetalization of D-glucose

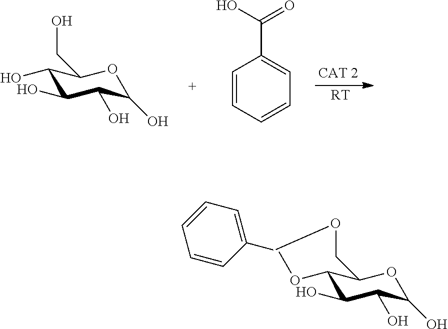

800 mg (4.44 mmol, 1 equiv) of D-glucose, 9010 mg (85 mmol, 19 equiv) of benzaldehyde and 300 mg of catalyst CAT 2 are introduced into a flask equipped with a magnetic bar. The mixture is stirred at ambient temperature for 16 hours. 15 mL of water is added, the solution is stirred for 1 hour, and then filtered on a frit. The solid obtained is washed with pentane and then dried under vacuum to produce a white powder. The filtrate is extracted with ether, this extract is washed with saturated NaCl solution, dried over MgSO₄, and then evaporated, to form a pale yellow solid. This solid and the white solid obtained previously are collected and recrystallized together from ethanol to form the expected product in the form of colourless needles.

### Example 9

### Formation of Imines

### Examples

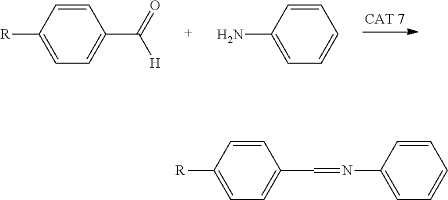 [0289] A variant of the reaction illustrates another possible strategy of the invention: the substrate is grafted on a solid phase. This is an approach complementary to heterogeneous catalysis.

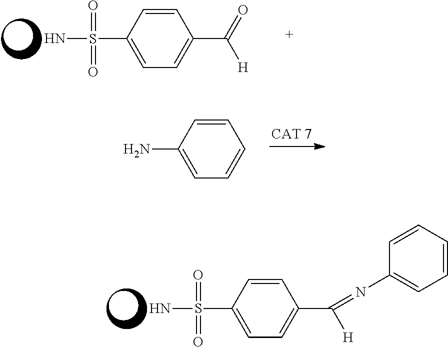 [SMILES: NS(=O)(=O)c1ccc(C=O)cc1 ; Nc1ccccc1 ; NS(=O)(=O)c1ccc(C=Nc2ccccc2)cc1]

The symbol:

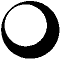

represents the solid support, which is a functionalized organic polymer.

The reaction may easily be extended to aliphatic aldehydes such as citronellal and to non-aromatic amines such as glucosamine. Examples are described for acridine structures and supporting with chitosan.

### Example 10

### Formylation of Amines

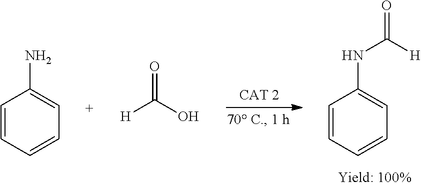 [SMILES: Nc1ccccc1 ; O=CO ; O=CNc1ccccc1]

In a typical procedure, 242 mg (2.6 mmol; 1 equiv) of aniline and 140 mg of catalyst CAT 2 (Zn content: 122,000 ppm) is introduced into a 25-mL round-bottomed flask equipped with a magnetic bar. While stiffing, 782 mg (15.6 mmol; 6 equiv) of formic acid is added in one go. The mixture is heated at 70° C. for one hour, with stirring. The reaction is complete in one hour.

### Example 11

### Concerted Rearrangements—Pericyclic Reactions

### Example 11a

### Ene-Reactions

### Example

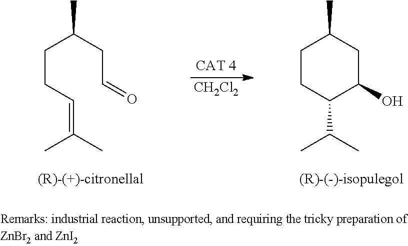

By adjusting the operating conditions, and notably by changing the solvent it is possible to transform the product of the ene-reaction, isopulegol, in situ to α-pinene, another natural product of industrial interest.

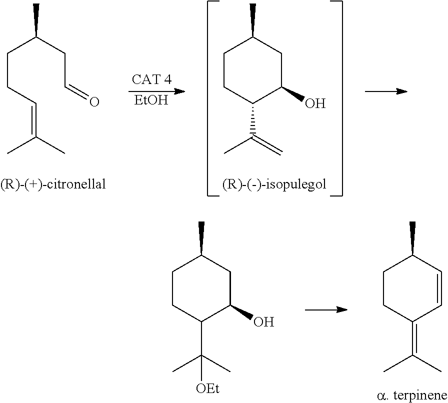

This possibility is specific to the catalysts derived from Sedum.
This evolution of isopulegol during its formation is unusual. This result reflects the greater acidity of the catalysts derived from S. plumbizincicola, making it possible to utilize the sequence carbonyl-ene reaction/ethanol addition/double elimination. It may be exploited for direct synthesis of α-terpinene, which is used in perfumery, cosmetics and as a food ingredient.

### Summary of the Operating Conditions:

| | | | % | % alpha- |
|---|---|---|---|---|
| CAT 4/citronellal/solvent | solvent | duration | isopulegol | terpinene |
| 100 mg/154 mg | CH₂Cl₂ | 1 h | 60 | 8 |
| (1 mmol, 1 equiv)/10 mL | | | | |
| 100 mg/154 mg | EtOH | 3 h | 10 | 80* |
| (1 mmol, 1 equiv)/10 mL | | | | |

*a trace of the intermediate product (ethanol addition) is detected in GC MS and LC MS
- Description of the preparation of isopulegol:

1 mmol of citronellal diluted in 10 mL of toluene is added to a 25-mL four-necked flask equipped with a CaCl₂ trap, a thermometer, a magnetized bar, a condenser and a dropping funnel. The catalyst CAT 4 (100 mg of catalyst with 68000 ppm of Zn, supported on 500 mg of montmorillonite K10, activated by heating at 150° C. for 15 minutes) is suspended in the solvent. The mixture is stirred for 60 minutes (the reaction is monitored by TLC (eluent: hexane/ether 4/1, I₂ developer)). The reaction mixture is filtered, and the organic phase is washed with a solution of sodium hydrogen carbonate, dried and concentrated. The yield and the stereoselectivity are determined by NMR and GC MS.

Description of the Preparation of α-Terpinene:

The process is similar to the previous one, but dichloromethane is replaced with ethanol and stirring is continued for 3 hours. The evolution of isopulegol to α-terpinene is easily monitored by GC MS and LC MS.

### Example 11b

### Cycloadditions: Examples of Diels-Alder Reactions

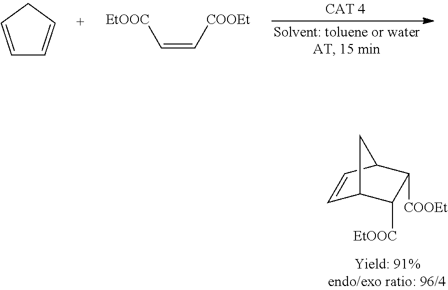 [SMILES: C1=CCC=C1 ; CCOC(=O)/C=C\C(=O)OCC ; CCOC(=O)C1C2C=CC(C2)C1C(=O)OCC]

The reaction may be carried out either in toluene or in water, an environmentally friendly solvent. In both cases, the cycloaddition products are obtained with yields of the order of 90% and with excellent selectivity (endo/exo ratio above 95/5).
In a typical procedure, 5 mL of solvent (toluene or water) is introduced into a 25-mL round-bottomed flask. The catalyst CAT 4 (100 mg of catalyst with 68,000 ppm of Zn, supported on 500 mg of montmorillonite K10, activated by heating at 500° C. for 15 minutes) is suspended in the solvent. 155 mg (0.9 mmol; 1.0 equiv) of diethyl maleate is added to the mixture. The mixture is stirred for 15 minutes. 145 mg (2.2 mmol; 2.44 equiv) of cyclopentadiene (obtained by distillation of dicyclopentadiene or 4,7-methano-3a,4,7,7a-tetrahydroindene) is then added in one go to the reaction medium, with stiffing. The mixture assumes a brick red colour after introduction of the cyclopentadiene, and then slowly turns brown. Samples are taken every 15 minutes for analysis by GC-MS.

### Example 11c

### Diels-Alder Cycloaddition with Asymmetric Induction Due to the Dienophile

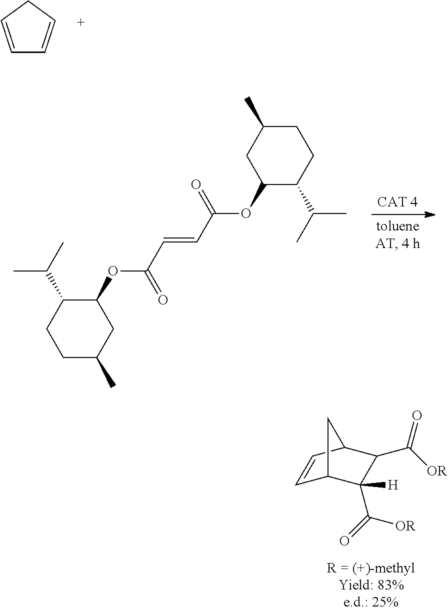

353 mg (0.9 mmol; 1.0 equiv) of dimenthyl fumarate is dissolved in 6 mL of anhydrous toluene, in a 25-mL flask, equipped with a magnetic bar. The catalyst of the CAT 4 type (100 mg of catalyst with 68000 ppm of Zn, supported on 500 mg of montmorillonite K10, activated by heating at 150° C. for 15 minutes) is suspended in the mixture. The medium is stirred for 15 minutes. 145 mg (2.2 mmol; 2.44 equiv) of cyclopentadiene (obtained by distillation of dicyclopentadiene or 4,7-methano-3a,4,7,7a-tetrahydroindene) is then added in one go to the reaction medium, with stirring. The mixture assumes a brick red colour after introduction of the cyclopentadiene, and then slowly turns brown. Samples are taken every 15 minutes for analysis by ¹H NMR (determination of d.e—diastereomeric excess—by comparing the chemical shift of the vinylic protons, which differs depending on the diastereoisomer). The cycloaddition product is obtained in 4 hours, with a yield of 83% and with a d.e of 25%.

### Example 11d

### Diels-Alder with Asymmetric Induction Due to the Chiral Support

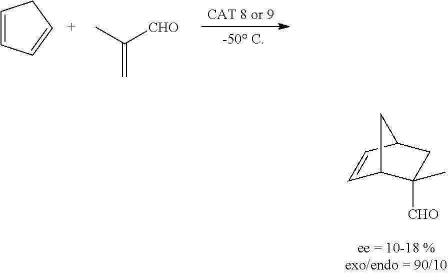

In a typical procedure, 5 mL of toluene is introduced into a 25-mL round-bottomed flask, at −50° C. The catalyst derived from Sedum supported on chiral organic polymer CAT 8 or CAT 9 (100 mg) is suspended in the solvent. 63 mg (0.9 mmol; 1.0 equiv) of methacrolein is added to the mixture. The mixture is stirred for 15 minutes. 145 mg (2.2 mmol; 2.44 equiv) of cyclopentadiene (obtained by distillation of dicyclopentadiene or 4,7-methano-3a,4,7,7a-tetrahydroindene) is then added in one go to the reaction medium, with stiffing. The medium assumes a brick red colour after introduction of the cyclopentadiene, and then slowly turns brown. The average yield is 75%.

### Example 11e

### Diels-Alder Reaction [4+2] Myrcene+Methyl Acrylate

The products of this reaction are related to several molecules used in the perfumery industry. It should be noted that a mixture of isomers is obtained, one of which is very predominant. The presence of several isomers may be of particular interest in perfumery, endowing the mixture with particular fragrance qualities. In particular, it is described in D. H. Pybus, C. S. Sell, Chemistry of Fragrances, RSC Publishing, Letchworth, 1999 that the presence of a cyclic isomer endows the mixture of products with a particular fragrance, for the same reaction starting from myrcene and from 3-methylpent-3-en-2-one.

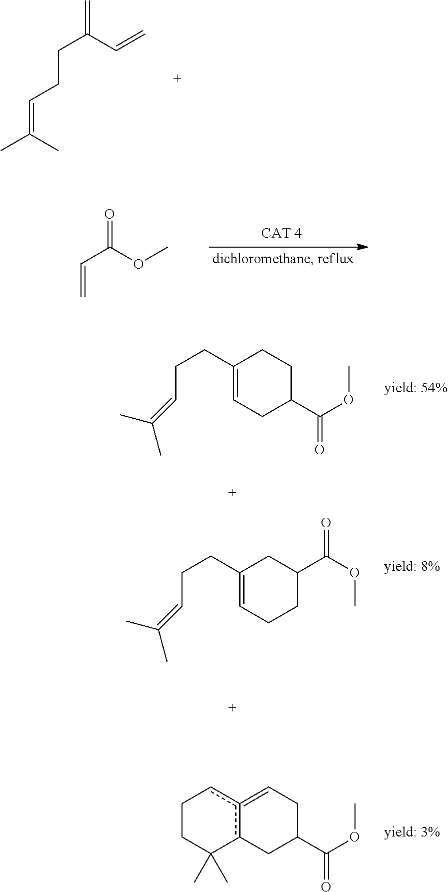

### Procedure:

The biosourced catalyst used in this reaction will be selected from the catalysts derived from Sedum plumbizincicola. The weight of catalyst used in the reaction is adjusted as a function of the catalyst's metal content, so that the reaction uses 10% (in mol of limiting reagent) of the specific metal species of the catalyst selected.
5 mL of dichloromethane and the weight of Lewis acid catalyst selected from the species mentioned above are introduced into a 10-mL flask, equipped with a magnetic bar and with a condenser, so that the reaction uses 10% (in mol of limiting reagent) of the specific metal species of the catalyst selected. 473 mg (5.5 mmol) of methyl acrylate is then introduced, the mixture is stirred for 5 minutes at RT, then 681 mg (5.0 mmol) of myrcene is added. The mixture is refluxed with stirring for 20 h, and then analysed by GC-MS.

### Example 110

### Synthesis of 2-Chromenes (Green Insecticides)

The 2H-chromenes of vegetable origin or precocenes constitute a new type of regulation of insect growth, and are becoming the ‘4th-generation insecticides’. They are regarded as non-ecotoxic and are perceived as biocontrol agents. The catalysts derived from Sedum allow direct and effective access to the precocenes.
The cascade reaction involves an electrophilic addition, dehydration of the adduct and an intramolecular cyclization of the hetero-Diels type.

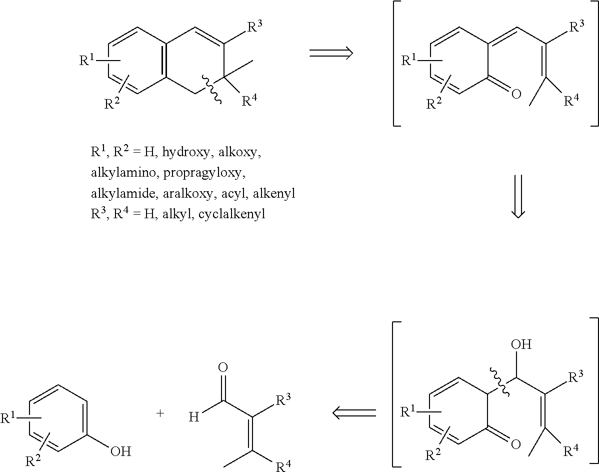

The method of synthesis is based on a reaction catalysed by biosourced catalysts of the CAT 4 type derived from Sedum plumbizincicola and supported on montmorillonite K10. The mixture of aldehyde and phenolic derivative is added to the catalytic system. The cascade reaction is activated by microwaves.
In a typical procedure, citral and sesamol are added to a mixture of CAT 4; the mixture is irradiated at 500 W for 8 minutes. The mixture is taken up in ethyl acetate, filtered on Celite and concentrated. The crude product is analysed by GC-MS, IR and NMR, and then purified by silica chromatography (Hexane/EtOAc: 9/1). The yield is 80%.

### Example 12

### Rearrangements

### Example 12a

### Opening of Epoxides

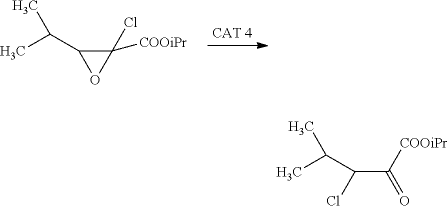

1 mmol of alpha-chlorinated glycidic ester is dissolved in 10 mL of anhydrous toluene, in a 25-mL flask equipped with a magnetic bar. The catalyst of the CAT 4 type (100 mg of catalyst with 68000 ppm of Zn, supported on 500 mg of montmorillonite K10, activated by heating at 150° C. for 15 minutes) is suspended in the mixture. The mixture is stirred for 60 minutes at 30° C., then filtered and concentrated. The isomerization product is obtained quantitatively and characterized by IR and ¹H NMR. Disappearance of the proton carried by the epoxide (3.42 ppm) and observation of the hydrogen at alpha of the ketone group (5.06 ppm) are characteristic of the proposed structure.

### Example 12b

### Pinacol Rearrangement

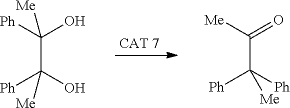 [SMILES: CC(O)(c1ccccc1)C(C)(O)c1ccccc1 ; CC(=O)C(C)(c1ccccc1)c1ccccc1]

0.5 mmol of the vicinal diol, diluted in 2 mL of toluene, is introduced into a 10-mL flask equipped with a water condenser connected in its turn to a CaCl₂ trap. The silica-supported Brønsted catalyst CAT 7 (1.56 mg of catalyst, or the equivalent of 0.04 mmol of ZnCl₂, is finely ground in the presence of 2.83 mg of SiO₂, then stirred in the presence of 5M HCl, concentrated and dried at 80° C.) is then added to the mixture, with stirring. The mixture is refluxed at 80° C. for 90 minutes.
The reaction is 60% effective in 90 minutes at 80° C. The reaction mixture is filtered, then evaporated and the catalyst is isolated and then dried for a subsequent reaction.
IR and then GC MS and ¹H NMR confirm the quantitative formation and purity of the product.

### Example 12c

### Beckmann rearrangement

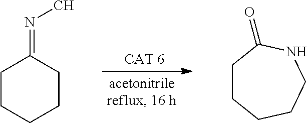

In a typical procedure, 5 mL of acetonitrile is introduced into a 25-mL round-bottomed flask equipped with a magnetic bar. 226 mg (2 mmol; 1 equiv) of cyclohexanone oxime is dissolved in this solvent, then 320 mg of catalyst of the CAT 6 type is added, with stiffing (catalyst with 61,000 ppm of Zn, supported on montmorillonite K10 protonated by treatment with 5M HCl for 3 hours). The mixture is heated under reflux for 16 hours. The reaction leads to 50% of rearranged product, the ε-caprolactam required for synthesis of nylon.

### Example 13

### Aldolization and Related Reactions

### Example 13a

### Claisen-Schmidt Reaction

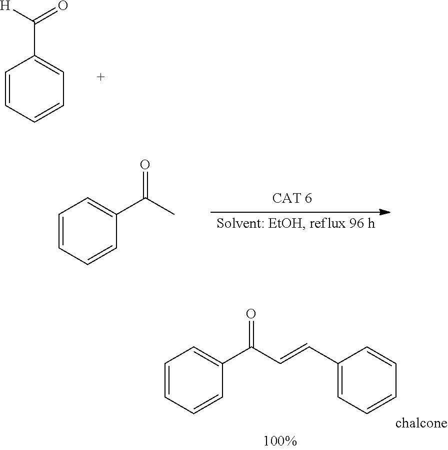

In a typical procedure, 110 mg of catalyst of the CAT 6 type (Zn content: 61,000 ppm) is introduced into a 25-mL round-bottomed flask equipped with a magnetic bar. 1 mL of ethanol is added, in order to suspend the catalyst. 106 mg (1.0 mmol; 1 equiv) of benzaldehyde and 120 mg (1.0 mmol; 1 equiv) of acetophenone are added to the mixture. The mixture is refluxed for 96 hours, with stirring. Chalcone is obtained with a yield of 100% in 96 hours.
Our catalytic system in particular allows the synthesis of industrially important compounds such as ionone:

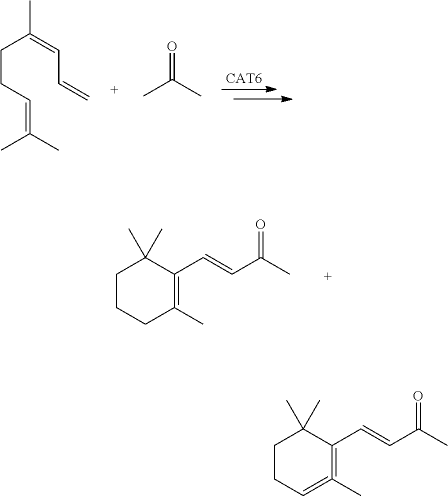

Two routes were explored, both leading to synthesis of ionone by aldolization of acetone and citral in acid catalysis:

a first route (A) consists of producing the enol from acetone progressively and reacting it with citral

a second route (B) utilizes a Mukaiyama aldolization, after in-situ synthesis of the silylated enol ether, in supported acid catalysis, using the catalysts derived from Sedum (for synthesis of the silylated enol ether, see the paragraph dealing with this step).

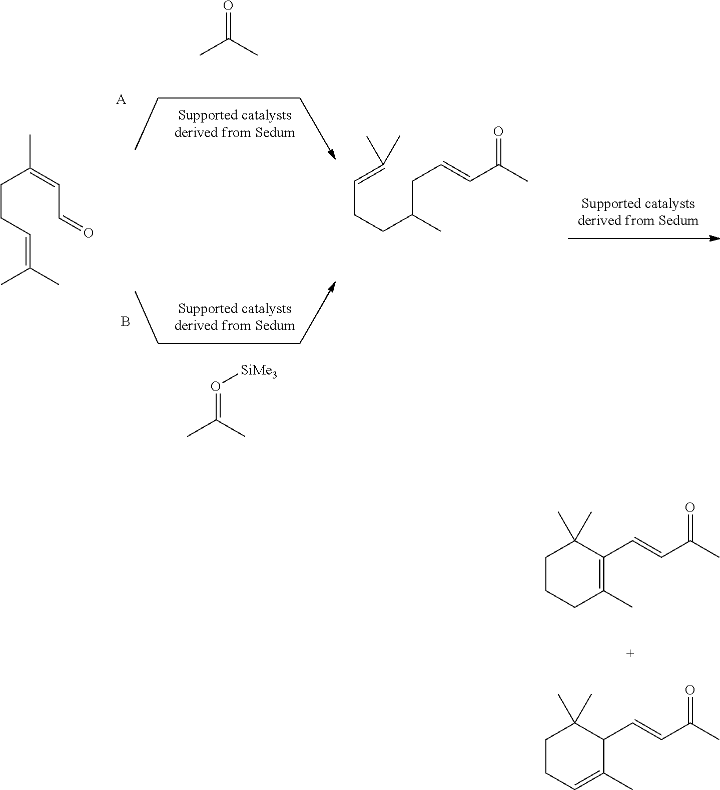

### Route A:

1522 mg (10 mmol, 1 equiv) of citral is diluted in 4 mL of ethanol, and introduced into a two-necked flask equipped with a magnetic bar and a condenser. 1500 mg of catalyst derived from Sedum is added to the mixture. The reaction mixture is stirred and heated to 60° C. Using a dropping funnel, 581 mg (10 mmol, 1 equiv) of acetone diluted in 5 mL of ethanol is added dropwise to the mixture, in 2 hours. Heating and stirring are continued after addition of the acetone, until there is formation of ionone.

### Route B:

20 mg of catalyst derived from Sedum, supported on 35 mg of silica, is introduced into a two-necked flask equipped with a magnetic bar and suspended in 5.4 mL of anhydrous acetonitrile. 174 mg (3 mmol, 1 equiv) of acetone is added, as well as 483 mg (3 mmol, 1 equiv) of hexamethyldisilazane. The medium is stirred for 2 hours, until the reaction is complete (monitored by infrared spectroscopy). The reaction medium is heated to 60° C., then using a dropping funnel, 456 mg (3 mmol, 1 equiv) of citral is added dropwise to the reaction medium in 2 hours. The mixture is stirred and heated until there is formation of ionone.

### Example 13b

### Mukaiyama Reaction

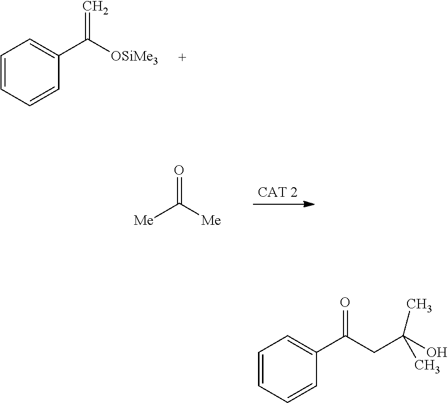

10 mL of dichloromethane and 1600 mg of catalyst CAT 2 (10 mmol of Zn) are added successively to a 50-mL three-necked flask, equipped with magnetic stirring, an isobaric funnel and an iced water bath. 11 mmol of acetone diluted in 15 mL of dichloromethane is added dropwise, then 1.92 g of enolsilylated ether derived from acetophenone at 0° C. The mixture is stirred for 30 minutes, and then filtered. The organic phase is washed with a solution of sodium hydrogen carbonate, dried over MgSO₄, filtered and concentrated. The crude product is purified by silica chromatography (eluent petroleum ether/ethyl acetate: 4:1, developer I₂—SiO₂)_{. The product is characterized by IR and} ¹H NMR. The yield is 68%.

### Example 13c

### Knoevenagel Reaction

### I.

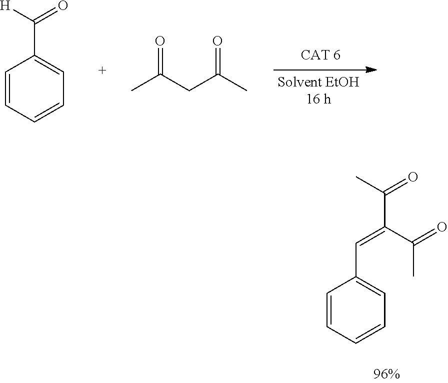

A second aldolization may take place after prolonged heating:

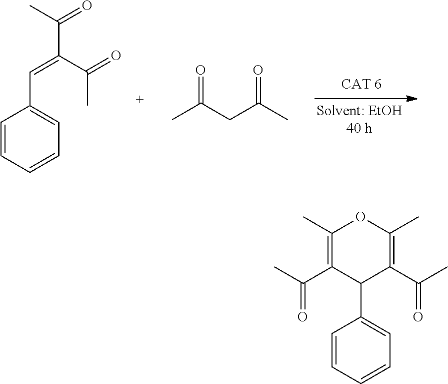

This reaction is described under acid catalysis but in noxious solvents such as toluene or hexane. The catalysts derived from plants of the Sedum type make it possible to carry out this reaction in ethanol, a non-toxic solvent that can be produced from biomass, which is in agreement with the principles of sustainable chemistry. The yields obtained using ethanol as solvent are, moreover, clearly greater than those found when using other solvents, such as dichloromethane (only 10% yield for the first aldolization in 16 hours).

### II. Another Knoevenagel Reaction

In a typical procedure, 100 mg of catalyst CAT 6 (Zn content: 122,000 ppm) is introduced into a 25-mL round-bottomed flask equipped with a magnetic bar. 1 mL of ethanol is added, in order to suspend the catalyst. 159 mg (1.5 mmol; 1 equiv) of benzaldehyde and 195 mg (1.5 mmol; 1 equiv) of ethyl acetoacetate are added to the mixture. The mixture is refluxed for 4.5 h, with stirring. The yield obtained is 90% in 4.5 hours.

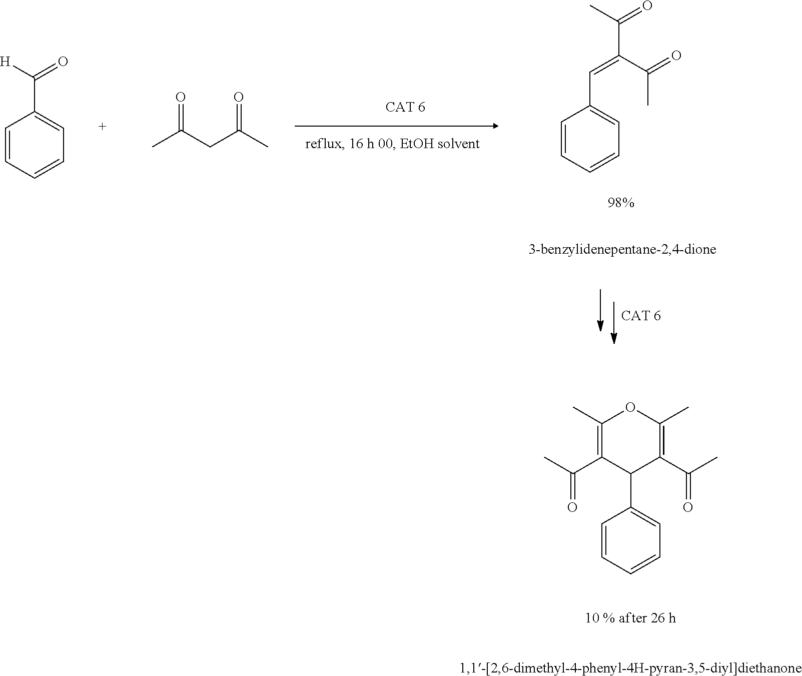

The reaction also takes place with acetylacetone, but a second aldolization is observed with prolonged heating, leading to formation of 1,1′-(2,6-dimethyl-4-phenyl-4H-pyran-3,5-diyl)diethanone.
In a typical procedure, 110 mg of catalyst CAT 6 (Zn content: 61,000 ppm) is introduced into a 25-mL round-bottomed flask equipped with a magnetic bar. 3 mL of ethanol is added, in order to suspend the catalyst. 106 mg (1.0 mmol; 1 equiv) of benzaldehyde and 250 mg (2.5 mmol; 2.5 equiv) of acetylacetone are added to the mixture. The mixture is refluxed for 16 h, with stirring. 3-Benzylidenepentane-2,4-dione is obtained with a yield of 98%. On continuing heating and stirring, 1,1′-(2,6-dimethyl-4-phenyl-4H-pyran-3,5-diyl)diethanone forms at a level of 10% in 26 hours.

### III. Reaction Cascade: Knoevenagel Reaction, Hetero-Diels-Alder Reaction[3+3], Diels-Alder Reaction[4+2]

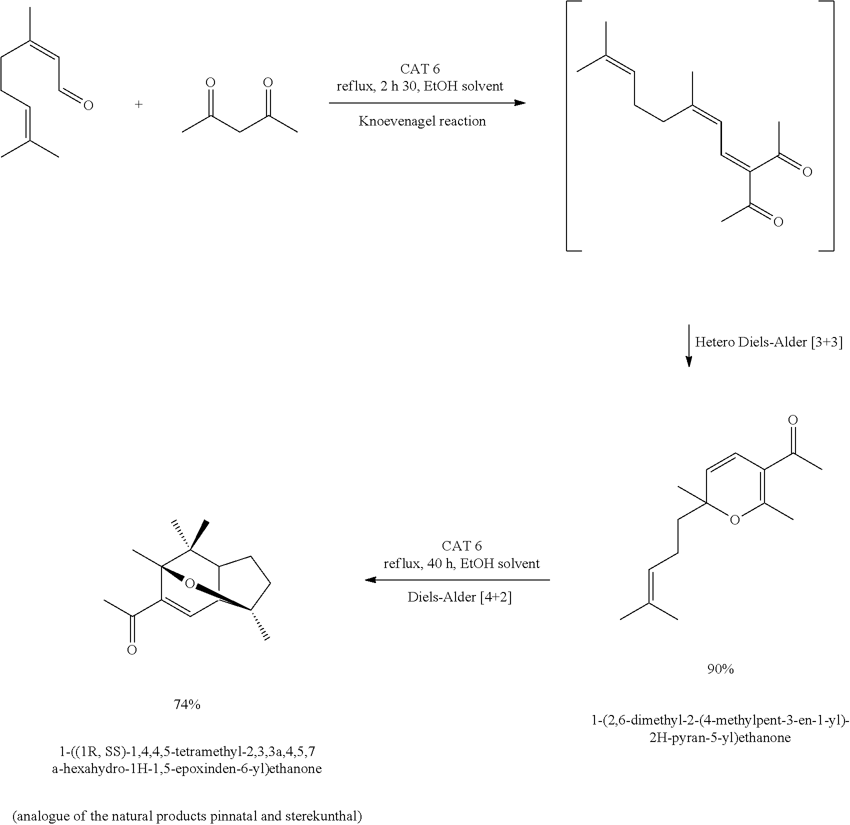

In a typical procedure, 110 mg of catalyst CAT 6 (Zn content: 61,000 ppm) is introduced into a 25-mL round-bottomed flask equipped with a magnetic bar. 3 mL of ethanol is added, to suspend the catalyst. 152 mg (1.0 mmol; 1 equiv) of citral and 100 mg (1.0 mmol; 1 equiv) of acetylacetone are added to the mixture. The mixture is refluxed for 2.5 h, with stirring. 142,6-dimethyl-2-(4-methylpent-3-en-1-yl)-2H-pyran-5-yl)ethanone is obtained with a yield of 89% after Knoevenagel and hetero-Diels-Alder reactions; the uncyclized intermediate could not be isolated. On continuing heating and stirring, 1-((1R,5S)-1,4,4,5-tetramethyl-2,3,3a,4,5,7a-hexahydro-1H-1,5-epoxyinden-6-yl)ethanone (analogue of the natural products pinnatal and sterekunthal) forms at a level of 74% in 40 hours.

### Example 14

### Dehydration

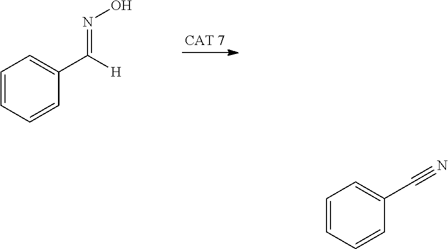 [SMILES: O/N=C/c1ccccc1 ; N#Cc1ccccc1]

1 mmol of benzaldehyde oxime dissolved in 10 mL of acetonitrile, 94 mg of catalyst CAT 7, and a few grains of molecular sieve are introduced into a two-necked flask equipped with a condenser, a CaCl₂ trap and a thermometer. The reaction is performed with reflux. It is completed after 3 hours of reaction. After filtration and concentration of the medium, IR and GC MS confirm quantitative formation of benzonitrile.

### Example 15

### Transfunctionalization

### Example 15a

### Transimination

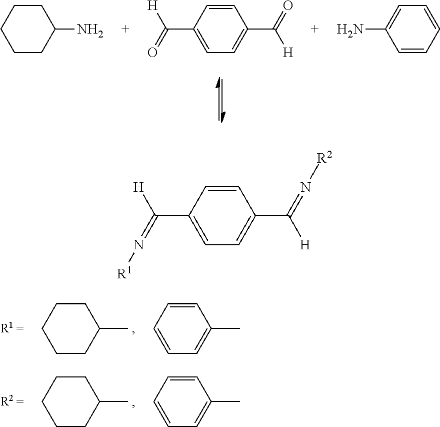

The principle of the reaction consists of guiding the reaction towards the formation of an aromatic imine or cycloalkyl depending on the plant used. Use of Sedum plumbizincicola or Sedum jinianum catalyse formation of the aromatic imine derived from aniline, whereas Potentilla griffithii promotes formation of the imine derived from cyclohexylamine. The results correlate directly with the level of zinc phytoextracted, and therefore ultimately the level of zinc present in the prepared catalyst. The exchange between imines is monitored by ¹H NMR. The proton signal characteristic of the aromatic imine is located at 8.7 ppm with an excess of catalysts derived from Sedum, whereas the same weight of catalyst derived from P. griffithii leads predominantly to the other imine detected at 8.4 ppm.

### Example 15b

### Transtritylation

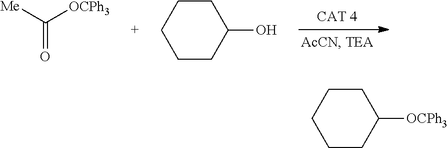

In a typical procedure, trityl acetate is freshly prepared according to the conditions described by Maltese et al. (2011, Tetrahedron Lett. 52, 483-487). Then, 1 mmol of cyclohexanol is added to the trityl acetate diluted in 5 mL of acetonitrile in the presence of 40 mg of catalyst derived from S. plumbizincicola CAT 4 dispersed on 71 mg of montmorillonite K10 (or 10% of Zn). The reaction is completed after stirring for 30 minutes at ambient temperature. IR and GC MS (internal standard: menthol) make it possible to confirm formation of trityl ether with 80% yield.

### Example 16

### Constructions of Simple and Complex Heterocycles

### Example 16a

### Preparation of Polyheterocyclic Structures

Preparation of Porphyrinogens

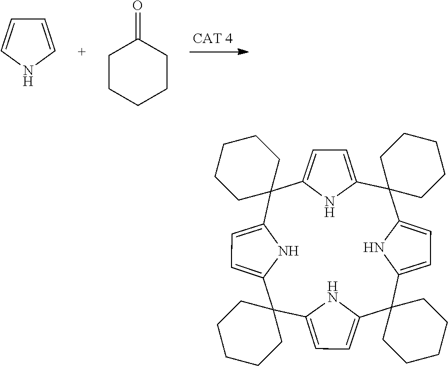 [SMILES: c1cc[nH]c1 ; O=C1CCCCC1 ; c1cc2[nH]c1C1(CCCCC1)c1ccc([nH]1)C1(CCCCC1)c1ccc([nH]1)C1(CCCCC1)c1ccc([nH]1)C21CCCCC1]

In a typical procedure, 200 μl (2.9 mmol 1 equiv) of pyrrole and 307 μL (2.9 mmol, 1 equiv) of cyclohexanone are introduced into a flask equipped with a magnetic bar. 1 mL of ethanol and 1 mL of water are added to the mixture, followed by slow addition of 1 g of catalyst CAT 4, in small portions. A milky suspension turning pink is formed, then a solid mass precipitates. The reaction medium is washed with water (3×50 mL) and then with dilute ammonia solution (1 M, 10 mL) and then ethanol (2×25 mL). The solid is then purified by recrystallization from hot acetone with slow addition of methanol and then cooling to ambient temperature. The product is obtained with a yield of 80%.
II. Preparation of (dithienyl)pyrroles

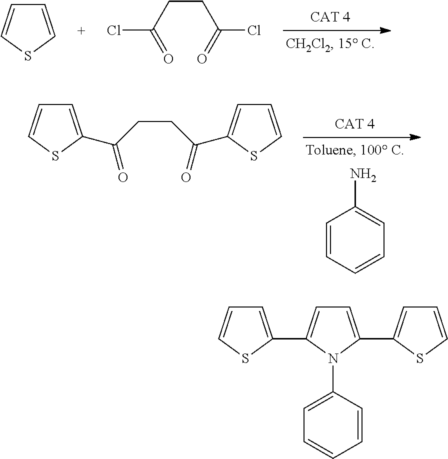

In a flask equipped with a magnetic bar, 168 mg (2 mmol, 2 equiv) of thiophene, 155 mg (1 mmol, 1 equiv) of succinyl chloride and 100 mg of supported catalyst CAT 4 are added to 5 mL of anhydrous dichloromethane. The mixture is stirred at 15° C. for 4 hours. The solvent is then removed by evaporation under reduced pressure, then 5 mL of toluene is added to take up the reaction product. The reaction medium is stirred again, then 93 mg (1 mmol, 1 equiv) of aniline is added. The mixture is heated at 100° C. for 24 h, until the reaction stops. The solvent is then evaporated and the product is purified by silica column chromatography (eluent: dichloromethane). The overall yield for the two steps is 60%.

### Example 16b

### Multicomponent Reactions

### Synthesis of Triazoles

### Synthesis of propargyl-1,2,3-triazoles

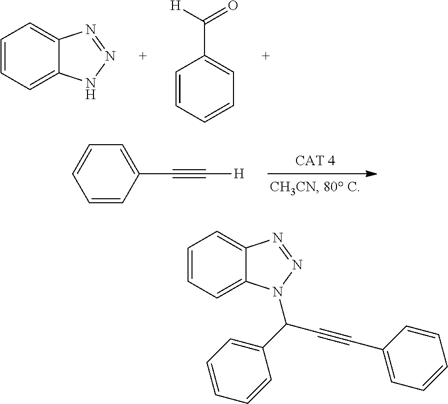 [SMILES: c1ccc2[nH]nnc2c1 ; O=Cc1ccccc1 ; C#Cc1ccccc1 ; C(#CC(c1ccccc1)n1nnc2ccccc21)c1ccccc1]

122 mg (1.2 mmol, 1.2 equiv) of phenylacetylene, 106 mg (1 mmol, 1 equiv) of benzaldehyde, 119 mg (1 mmol, 1 equiv) of benzotriazole and 100 mg of supported catalyst CAT 4, in 2 mL of acetonitrile, are introduced into a flask equipped with a magnetic bar. The reaction medium is heated at 80° C. with stirring, for 10 hours. Once the reaction is completed (monitored by TLC, eluent hexane/EtOAc 9/1), the reaction medium is concentrated by evaporation under reduced pressure and is then taken up in 3×10 mL of EtOAc and filtered. The resultant organic phase is dried over Na₂SO₄ and is then evaporated under reduced pressure. The residue obtained is purified by silica column chromatography (eluent hexane/EtOAc 20/1). It is obtained with a yield of 85%.

Hantsch and Related Reactions

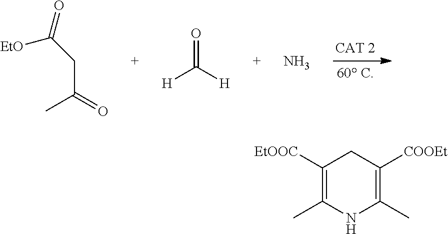

300 mg of paraformaldehyde (10 mmol, 1 equiv), 2600 mg (20 mmol, 2 equiv) of ethyl acetoacetate, 1540 mg (20 mmol, 2 equiv) of ammonium acetate and 50 mg of catalyst CAT 2 are introduced into a flask equipped with a magnetic bar. The mixture is heated at 60° C., with stirring, for 1 hour. Once the reaction is completed (monitored by TLC), the reaction medium is diluted with EtOAc (20 mL), the organic phase is washed with a saturated sodium hydrogen carbonate solution (3×20 mL), then saturated NaCl (1×20 mL). The organic phase is dried over anhydrous Na₂SO₄ and then concentrated under reduced pressure, to give the crude product, which is purified by recrystallization from EtOAc/ether 1/1 mixture. The yield is of the order of 90%.

Biginelli Reaction

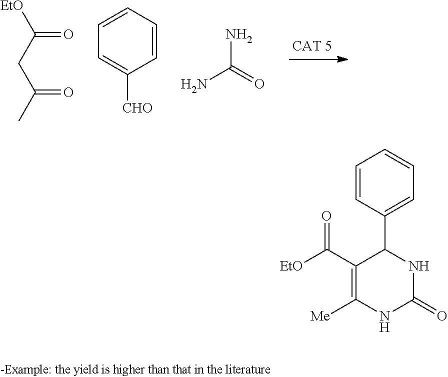

235 mg of catalyst CAT 5 derived from Sedum plumbizincicola dispersed on 425 mg of silica, then 2.5 mmol of benzaldehyde, and 2.5 mmol of ethyl acetoacetate and 1.25 mmol of urea in 15 mL of acetonitrile are introduced into a flask equipped with a magnetic bar, a condenser, a feed funnel and a thermometer. The mixture is taken to reflux for 12 hours. The reaction is monitored by TLC (UV detection, eluent: diethyl ether), then the mixture is filtered and the filtrate is concentrated. The crude product is purified by crystallization from EtOAc-hexane mixture, and then analysed by ¹H NMR, ¹³C NMR, COSY, HSQC and IR. The yield reaches 70%.

Synthesis of Piperidines and Substituted Piperidines

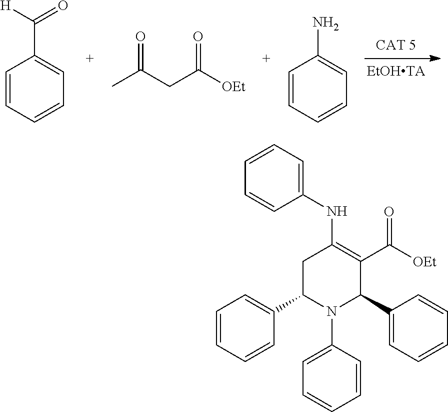

186 mg (2 mmol, 2 equiv) of aniline, 130 mg (1 mmol, 1 equiv) of ethyl acetoacetate and 100 mg of catalyst of the CAT 5 type in 4 mL of ethanol are introduced into a flask equipped with a magnetic bar. The mixture is stirred at ambient temperature for 20 minutes, then 212 mg (2 mmol, 2 equiv) of benzaldehyde is added, and stirring is continued until completion of the reaction, i.e. for approximately 18 hours. A precipitate is obtained, which is recovered by filtration and then washed with water/ethanol 1/1 mixture at 0° C. The solid is dissolved in 10 mL of a hot ethyl acetate/ethanol mixture (50° C.), filtered in order to remove the catalyst, and then the filtrate is left to cool slowly so that the reaction product crystallizes. The yield reaches 60%.

### Example 17

### Biomimetic Reductions and Transfers of Hydrides

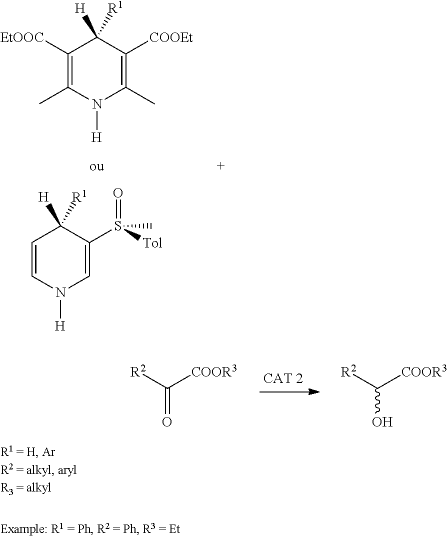

NADH is Nicotinamide-Adenine Dinucleotide H, it is a natural hydride donor that carries out reduction reactions by hydride transfer in all living cells. Here, the reaction is not catalysed by NADH dehydrogenase, but by a catalyst derived from SEDUM; the reaction is the same, but the catalyst is synthetic and not enzymatic.
This constitutes an advantage as a catalyst such as Sedum is of more general use than an enzyme.
In a typical procedure, 2 mmol of dihydropyridine diluted in 20 mL of toluene is introduced into a 100-mL flask placed under inert atmosphere. 110 mg of catalyst CAT 2 (Zn content: 61,000 ppm) is added to the reaction medium. After stirring for 5 minutes, ethyl phenylpyruvate (2 mmol) diluted in 15 mL of toluene is added dropwise. The reaction is monitored by TLC (average reaction time: 90 minutes). Once all the pyruvate has been consumed, the solution is filtered, and the solvent is evaporated. Purification by column chromatography leads to 80% of hydroxy ester (GC MS purity) with an enantiomeric purity of 94%.

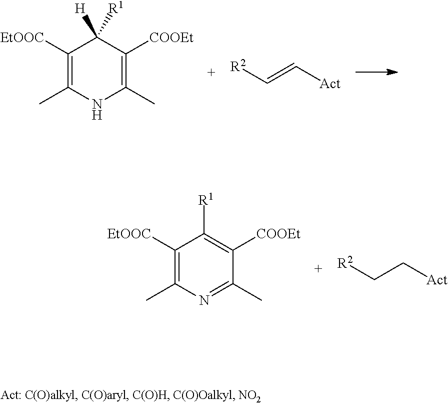

In the same spirit of a biomimetic approach, it is possible to reduce a double bond if it is activated by an electron-attracting group. An illustrative example is the chemoselective reduction of nitrostyrene. The carbon-carbon double bond is hydrogenated without risk of reduction of the nitro group.

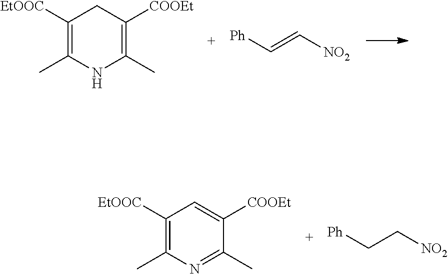 [SMILES: CCOC(=O)C1=C(C)NC(C)=C(C(=O)OCC)C1 ; O=[N+]([O-])/C=C/c1ccccc1 ; CCOC(=O)c1cc(C(=O)OCC)c(C)nc1C ; O=[N+]([O-])CCc1ccccc1]

In a typical procedure, 2 mmol of dihydropyridine diluted in 20 mL of dichloromethane is introduced into a 100-mL flask placed under inert atmosphere. 110 mg of catalyst CAT 2 (Zn content: 61,000 ppm) is added to the reaction medium. Nitrotoluene (2 mmol), diluted in 15 mL of dichloromethane, is added, and then the medium is concentrated. After 4 minutes of microwave irradiation at 150 W, the solution is cooled and then filtered. The organic medium is washed with concentrated HCl solution, dried and the solvent is evaporated. Purification by column chromatography leads to 75% of 2-phenylnitroethane (GC MS purity).

### Example 18

### Isomerization

The migration of a double bond under thermodynamic control can be catalysed quantitatively by CAT 10 or CAT 14.

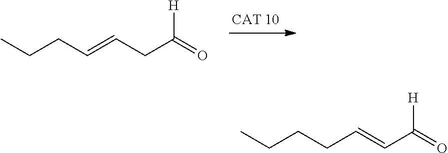

Operating conditions: 50 mg of 3-hexenal is diluted in 2 mL of EtOH under a nitrogen atmosphere. 100 mg of CAT 10 is added with stirring. The reaction is monitored by IR (shifts of the vibration band of the C═C double bond from 1659 to 1637 cm⁻¹, and of the C═O double bond from 1726 to 1683 cm⁻¹). After stirring for 3 hours, the reaction mixture is filtered and the medium is evaporated under nitrogen. The reaction is complete (monitoring with IR and ¹H NMR).

### Part 3

### Lewis Acid Cocatalysis During Functional Transformations by Reduction of a Transition Metal

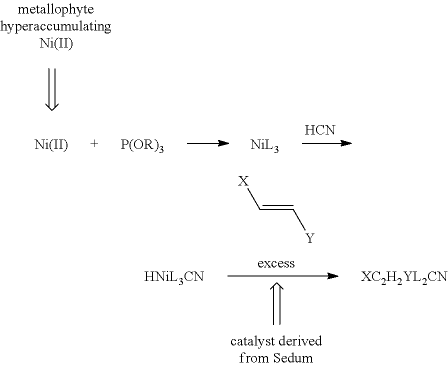

1. Investigation of the first step: preparation of an organonickel compound starting from metallophyte species hyperaccumulating Ni(II).

Nickel of oxidation state zero is an efficient reagent for elongating the carbon skeleton of an aryl or of a vinyl while avoiding the magnesia or multistep routes, which are unsuitable for the current principles of green chemistry. Preparation of an active catalyst of metallophyte origin is described below for the first time, with two illustrative examples, the preparation of arylphosphonates and the Heck reaction. The results prove reduction of Ni(II) of vegetable origin by a phosphite to the active entity Ni(0).

### Example 1

### Preparation of Arylphosphonates

In a 10 mL four-necked flask placed under an inert atmosphere, and equipped with a magnetic stirrer, a thermometer, a condenser and an isobaric funnel, 0.4 mmol of triphenylphosphite and 0.9 mmol of iodobenzene are added to 20 mg of catalyst derived from Psychotria douarrei (Ni 178,000 ppm) as prepared by the process described in example 5.2 of application WO 2011/064487. The reaction mixture is heated to 150° C. 0.2 mmol and 0.315 mmol of triethylphosphite are gradually added to the reaction mixture, which is heated and stirred for 4 hours. The reaction is monitored and characterized by ³¹P NMR ((PhO)₃P: 138 ppm; (EtO)₃P: 140 ppm; (PhO)₂P(O)Ph: 12 ppm). The solution is filtered. The IR, ¹H NMR and ¹³C NMR data confirm formation of the expected phosphonate.

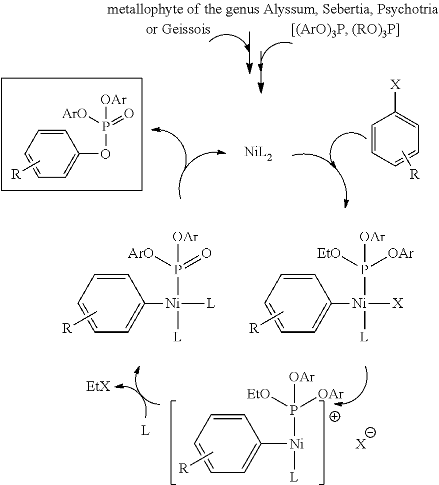

### Example 2

### Reaction of the Heck Type: The Reaction was Carried Out Between Iodobenzene and Styrene

In a 10 mL four-necked flask placed under inert atmosphere, and equipped with a magnetic stirrer, a thermometer, a condenser and an isobaric funnel, 1 mmol of iodobenzene, 2 mmol of styrene and 2 mmol of potassium carbonate are added to 20 mg of catalyst derived from Psychotria douarrei (Ni 178,000 ppm). 5 mL of methylpyrrolidinone is added, then the reaction mixture is heated at 150° C. for 24 hours. After filtration, washing with water, extraction with ethyl acetate, drying and concentration, the crude product is purified by silica chromatography. The data from GC MS, IR and ¹H and ¹³C NMR confirm formation of the coupled product with a yield of 80%.

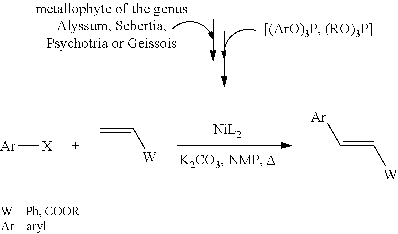

2. Investigation of the second step: hydrocyanation of alkenes cocatalysed by metallophyte species that are hyperaccumulators of Ni(II) such as Psychotria douarrei and of Zn(II), such as from the genus Sedum, preferably Sedum plumbizincicola.
The intermediate catalytic species of the reactions successively utilized may be characterized by NMR and IR spectroscopy. Reduction of Ni(II) to Ni(0) is carried out with a tritolylphosphite (designated L hereafter) at 60° C. according to the principle described in the previous paragraph. The solution is cooled to −78° C. Bubbling of HCN in the reaction medium rapidly leads to a yellow coloration, providing evidence of formation of the key species HNiL₃CN. This conclusion is supported by observation of an IR band characteristic of the CN vibrator 2125 cm⁻¹, which is shifted to 2174 cm⁻¹ after addition of an extract of catalyst of the CAT 3 type. This result is in agreement with formation of the mixed catalytic species HNiL₃CN, ZnCl₂.

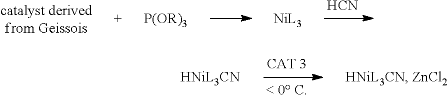

Controlled formation of the final mixed species HNiL₃CN, ZnCl₂ allows alkyldinitriles to be prepared by cocatalysis with the Zn-hyperaccumulating species of the genus Sedum and in particular Sedum plumbizincicola:

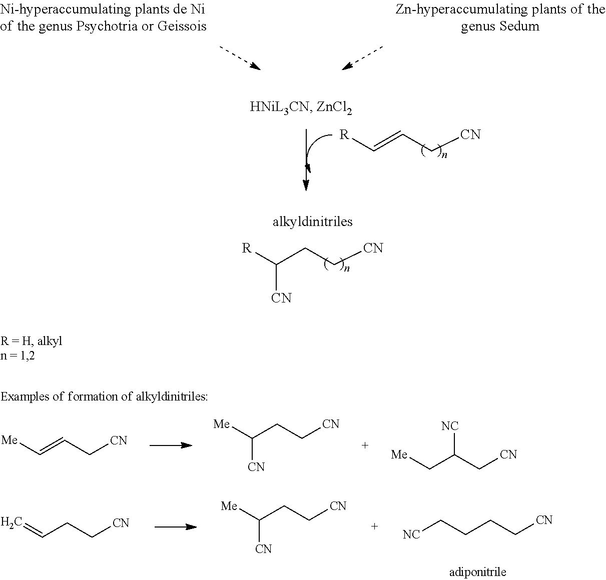

The process for preparation of alkyldinitrile consists of adding alkenenitrile to the catalytic complex HNiL₃CN, ZnCl₂ in the following molar proportions: alkenenitrile/triarylphosphite/Ni(P(OAr)₃/CAT 3 (Zn)/HCN: 25/0.8/0.1/0.2/130 mmol

### Part 4

### Multistep Syntheses Based Exclusively on the Organic Catalysis of Vegetable Origin where the Lewis Acid Properties of the Catalysts Derived from Sedum Play a Key Role

### Example 1

### Chloromethylation/Cyanation and Hydrochlorination/Cyanation Starting from Products of Vegetable Origin (Green Chemistry)

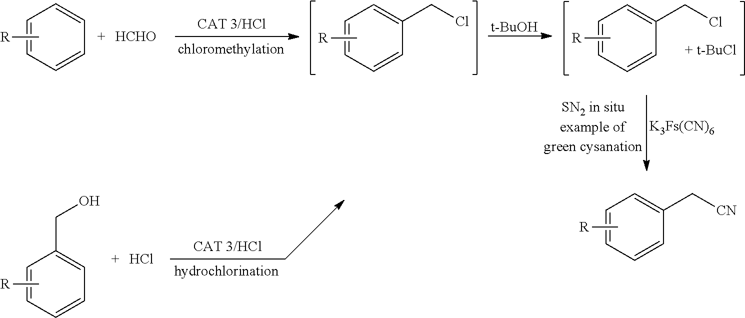

### Example

This multistep sequence, which is carried out in situ, without isolating and therefore without purifying the reaction intermediates, was developed on several examples of aromatic structures (derivatives of benzene and of naphthalene).
The processes described for the benzene series are strictly transposable to the naphthalene series.
Chloromethylation of toluene:
   25 mmol of toluene, 5 mmol of paraformaldehyde and 500 mg of catalyst CAT 3 containing between 5 and 15% of Zn are added successively to a 25-mL two-necked flask containing 5 mL of 4M hydrochloric acid. The mixture is stirred vigorously and heated at 60° C. for 8 hours. It is used directly in the next step.
Hydrochlorination of benzyl alcohol:
   2 mmol of alcohol is added at 25° C. to ? g of a catalyst CAT 3 in solution in 12M HCl. The mixture is stirred for 3 hours at 25° C. The chlorinated derivative is not isolated.
Neutralization of the excess hydrochloric acid with tert-butanol and formation of tert-chlorobutane: The acidity of the mixture is neutralized by adding a stoichiometric quantity of tert-butanol relative to the quantity of hydrochloric acid used either during chloromethylation, or during hydrochlorination.
Green cyanation: After stirring for 2 h, 5 mmol of potassium ferrocyanide is added in small portions. The reaction is completed after 12 hours of stirring at 40° C. The filtrate is washed with water, dried and concentrated. The cyanation product is inspected by IR and then GC MS.

### Example 2

### Protection/Selective Deprotection

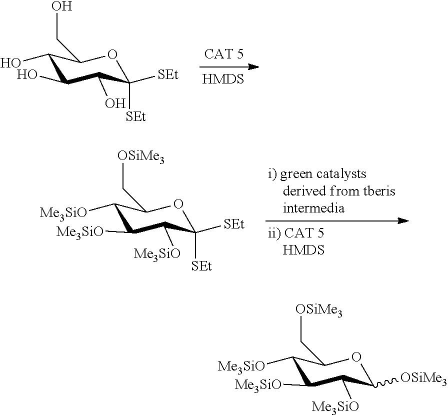

i) Complete silylation of D-glucose diethyl mercaptan:
The nucleophilic substrate (1 mmol) is introduced into a 25-mL flask equipped with a magnetized bar for magnetic stirring and a CaCl₂ trap. 0.75 equivalent of hexamethyldisilazane per alcohol to be silylated, i.e. 3 mmol diluted in 5 mL of acetonitrile, is added. The silica-supported catalyst CAT 5 (47 mg of catalyst is ground finely, i.e. the equivalent of 0.12 mmol of ZnCl₂, in the presence of 85 mg of SiO₂, then dried by heating on an electric heater for 15 minutes at 150° C.) is then added to the mixture, with stirring. The reaction is complete in 25 minutes at ambient temperature. The reaction mixture is filtered, then evaporated and the catalyst is isolated and then dried for a subsequent reaction.

ii) Deblocking of the dithio acetal unit and subsequent silylation:

The catalyst is prepared by the following process:
1. Leaves of Iberis intermedia (T1-accumulating plant, see WO 2011/064487) are dried after harvesting, ground and treated thermally at 400° C. for 5 hours in order to destroy the organic matter. The composition of the ash obtained (percentage by weight) is as follows:

| | Mg | Al | Ca | Zn | As | Cd | Tl | Pb |
|---|---|---|---|---|---|---|---|---|
| I. intermedia | 7.767 | 1.557 | 26.175 | 1.932 | 0.002 | 0.021 | 0.120 | 0.179 |

2. 325 mg of ash obtained in 1 is treated with 6.5 mL of concentrated nitric acid (65% HNO₃) at 60° C. for 2 hours with magnetic stirring.
3. The solution obtained is then filtered on Celite and concentrated under reduced pressure before being used in organic synthesis.
   The nucleophilic substrate (0.5 mmol), together with 6 mL of acetonitrile, is introduced into a 10-mL flask equipped with a magnetized bar for magnetic stirring. 500 mg of catalyst is finely ground, then dried by heating on an electric heater for 15 minutes at 150° C. and is then added to the mixture, with stirring. The reaction is 15% effective in 22 hours at ambient temperature. The product is silylated directly and is analysed by GC MS. For this, the silylation procedure described previously is followed.
   The reaction mixture is filtered, then evaporated and the catalyst is isolated and then dried for a subsequent reaction.
   The crude product is analysed by ¹H NMR, ¹³C NMR, COSY, HSQC and HMBC. The persilylated glucose is obtained in the form of the two anomers, alpha and beta, easily identifiable in ¹H NMR: 2 doublets respectively at 5.7 and 5.5 ppm in the ratio 66/33. The ease and the chemoselective conditions of deprotection of the dithioacetal unit should be noted.
   The process is easily transposed to thio acetal.

### Example 3

### Depolymerization/Garcia Gonzalez Reaction/Chemoselective Protection or Selective Oxidation

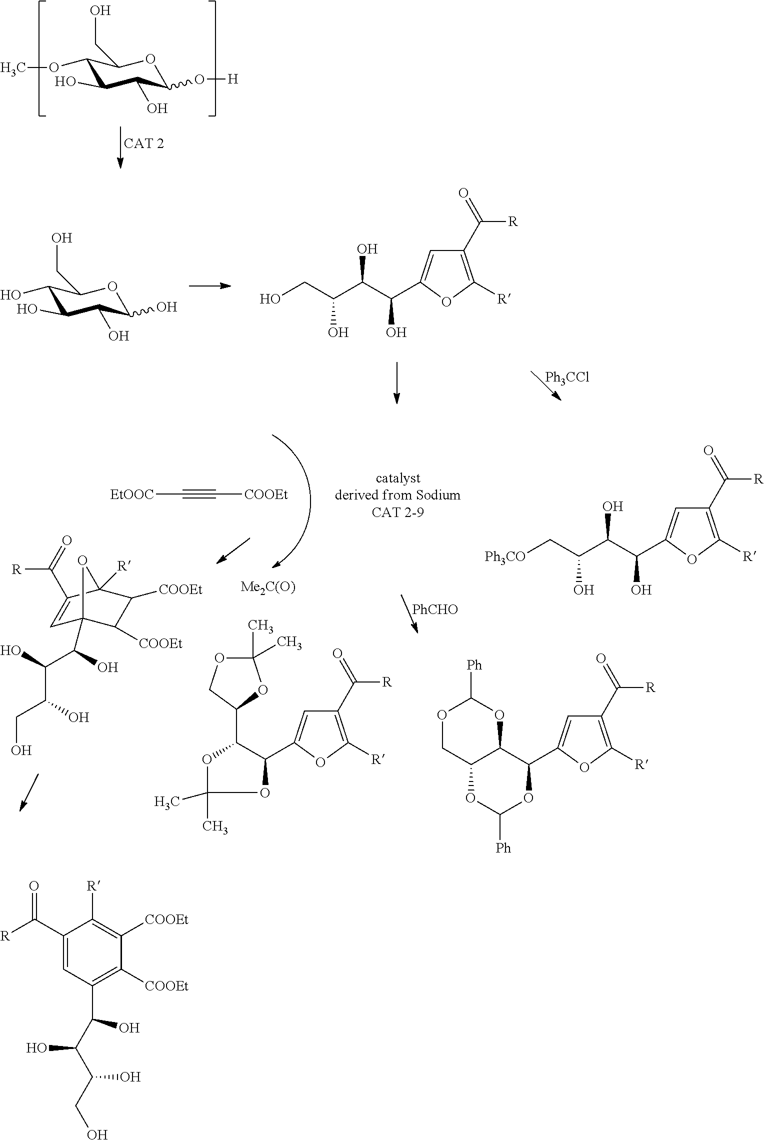

R,R′: alkyl, ester for example ethyl ester
The reaction utilizes a succession of transformations between a hexose and a dicarbonylated compound. The hexose is in particular obtained after depolymerization of cellulose (to glucose) by means of the CAT 2 catalysts.

### Depolymerization of Cellulose to Glucose:

10 mL of water and 1500 mg of catalyst CAT 2 are added to 1 g of cellulose in a flask equipped with a magnetic bar. The mixture is stirred at 65° C. for 1 hour. After this stirring, the mixture is poured into 50 mL of ethanol and a zinc-cellulose precipitate is formed. The latter is filtered and washed with water (40 mL) and ethanol (20 mL), and then dried under reduced pressure. A very dilute aqueous solution of hydrochloric acid is then added in order to complete the hydrolysis of the cellulose: the precipitate is brought into contact with 10 mL of hydrochloric acid at 0.1% (weight/volume) and the mixture is heated to 85° C. After reaction for 24 h, hydrolysis is 90% effective.
The reaction also takes place with the following sugars: mannose, ribose, lyxose, arabinose, xylose and with the following dicarbonylated compounds: ethyl acetoacetate, cyclohexanedione, 2-hydroxy-1,4-naphthoquinone, dimedone.
Procedure for the Garcia Gonzalez Reaction with Ethyl Acetoacetate and Glucose:

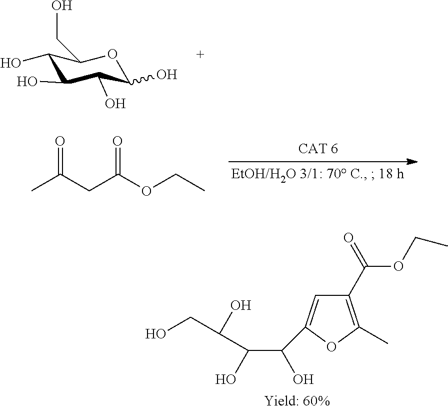

### Reaction Conditions:

1.5 mL of ethanol and 0.5 mL of water are introduced into a 25-mL flask.
1000 mg of anhydrous glucose (5.55 mmol, 1.40 equiv) and 500 μL of ethyl acetoacetate (3.95 mmol, 1.0 equiv) are added, with magnetic stirring.
450 mg of Lewis acid/Brønsted acid catalyst (CAT 6, with 61,000 ppm of Zn) is added to the mixture (i.e. 0.40 mmol of Zn, 0.10 equiv).
The mixture is heated on an oil bath, at 70° C. in the bath. Stirring and heating are maintained for 18 hours. The progress of the reaction may be monitored by TLC with the eluents toluene/acetone 1/1 (product Rf=0.1) or DCM/MeOH 9/1 (Rf not calculated). The mixture is dark red owing to formation of a complex between the enol form of ethyl acetoacetate and the transition metals of the catalyst, in particular iron.

### Purification:

Two techniques are possible, either by liquid/liquid extraction and then recrystallization, or by hot filtration and then recrystallization (this second method, being more economical of solvent and quicker, is preferably selected).

1) Liquid/Liquid Extraction

At the end of reaction, the mixture is taken up in EtOAc (30 mL) and water (30 mL), to dilute any solid residues. If these residues persist, filter the mixture on a frit and take up the solid residues in hot EtOAc, as the residues may contain Garcia Gonzalez product that has precipitated. (It is important that the EtOAc is hot, near boiling, as the reaction product has quite low solubility in cold EtOAc).
Extract the aqueous phase with EtOAc, optionally after stirring the aqueous phase in the presence of EtOAc on a heating plate (at 50° C.) as extraction of the product with EtOAc is mediocre. Repeat the extraction for as long as the aqueous phase contains the product (check by TLC). Usually 500 mL of EtOAc is necessary for good extraction (repeated 5 times or more).
The organic phase is washed with saturated NaCl (aq) and then dried over anhydrous MgSO₄. It is evaporated under reduced pressure to give a white solid, consisting almost exclusively of the pure Garcia Gonzalez product (the yield may be estimated on this crude mass).

2) Hot Filtration

At the end of reaction, the flask contents are taken up in acetone and the minimum of water and transferred to a large flask. The mixture is evaporated to dryness under reduced pressure, without exceeding 70° C. in the bath (same heating conditions as the reaction).
The traces of water are removed by co-evaporation in the presence of a large excess of toluene (repeat azeotropic evaporation 2 or 3 times).
The solid residue is taken up in boiling EtOAc (stirring in the bath of the rotary evaporator at atmospheric pressure). The liquid obtained is filtered hot on a frit, repeating the operation 2 or 3 times, until the EtOAc phase no longer contains reaction product after stirring while hot.
The organic phase is evaporated under reduced pressure; a yellowish solid is obtained. The latter is washed with cold hexane (or cold dichloromethane); a white solid remains at the bottom of the flask, constituted by the reaction product of good purity.

3) Recrystallization

The product may be purified by recrystallization, by taking up the white solid in hot EtOAc. Recrystallization is fairly quick and easy; fine white needles are deposited by slow cooling to ambient temperature.
The yield of the reaction is 60%, which is above the values described in the literature in the liquid phase.
Procedure for the Garcia Gonzalez Reaction with Acetylacetone and Glucose:

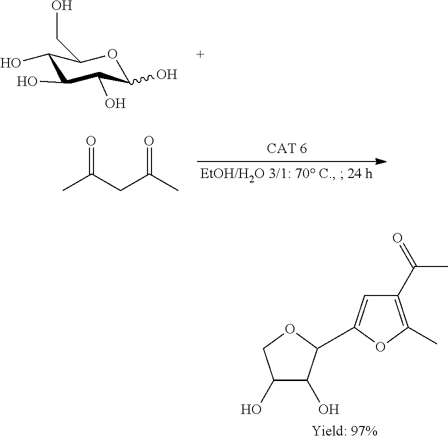

### Reaction Conditions:

1.5 mL of ethanol and 0.5 mL of water are introduced into a 25-mL flask.
1000 mg of anhydrous glucose (5.55 mmol, 1.40 equiv) and 395 mg of acetylacetone (3.95 mmol, 1.0 equiv) are added, with magnetic stirring.
450 mg of Lewis acid/Brønsted acid catalyst (CAT 6, with 61,000 ppm of Zn) are added to the mixture (i.e. 0.40 mmol of Zn, 0.10 equiv).
The mixture is heated on an oil bath, at 70° C. in the bath. Stirring and heating are maintained for 24 hours. The progress of the reaction may be monitored by TLC with the eluents toluene/acetone 1/1 (product Rf=0.1) or DCM/MeOH 9/1 (product Rf=0.7). The mixture is dark red because of formation of a complex between the enol form of ethyl acetoacetate and the transition metals of the catalyst, in particular iron.

### Purification:

At the end of reaction, the reaction mixture is evaporated to dryness under reduced pressure. Azeotropic co-evaporation with toluene is carried out to remove the traces of water that remain. A viscous brown product is obtained. The latter is adsorbed on silica gel (5 g) after dilution in methanol (10 mL) and then separated by silica column chromatography (30 g, elution dichloromethane/methanol 8/2). The product is developed with KMnO₄ in TLC. The product of the Garcia Gonzalez reaction is obtained with a yield of 97%, which is slightly higher than the best values described in the literature.
(Nagarapu, L.; Chary, M. V.; Satyender, A.; Supriya, B.; Bantu, R., Iron(III) Chloride in Ethanol-Water: Highly Efficient Catalytic System for the Synthesis of Garcia Gonzalez Polyhydroxyalkyl- and C-Glycosylfurans. Synthesis 2009, 2009 (EFirst), 2278, 2282).

### Other Examples of Garcia Gonzalez Reaction

The biosourced catalysts derived from metal-accumulating plants of the genus Sedum or from the plant Potentilla griffithii have allowed catalysis of the Garcia Gonzalez reaction with a large number of different substrates, leading to a large variety of products. The reaction has in particular been carried out starting from glucose and glucosamine, leading to furan and pyrrole respectively. An oxygen-sulphur exchange was carried out from the previous furan, leading to substituted thiophene in the same way as the original furan.
Variations of dicarbonylated compound have also been produced, by using the following compounds: ethyl acetoacetate, acetylacetone, cyclohexane-1,3-dione.

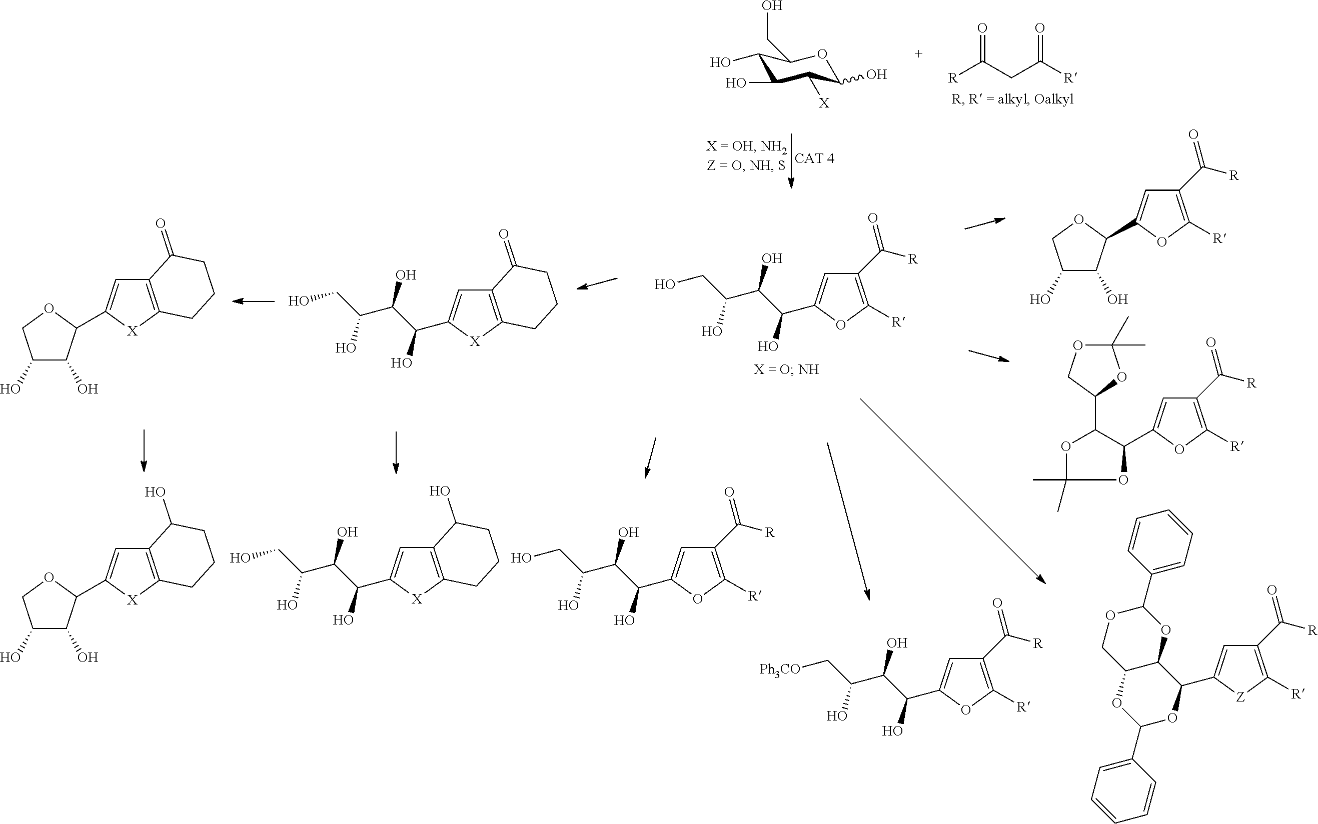

Variations of sugar were produced; all the reaction sequences presented in the above diagram proceeded starting from the following sugars: glucose, mannose, rhamnose, xylose, ribose, glucosamine, having in consequence a shortening or a modification of stereochemistry of the polyhydroxyl chain.

| Starting sugar | Reaction product | Yield |
|---|---|---|
| 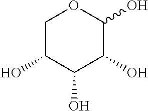 | 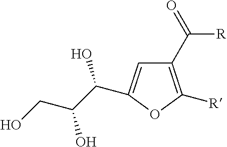 | 70% |
| 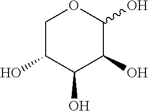 | 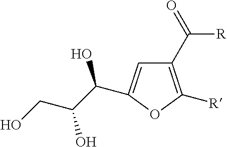 | 72% |
| 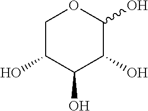 | 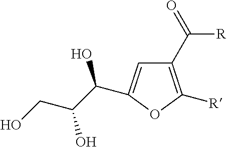 | 70% |
| 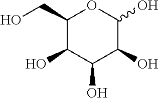 [SMILES: OC[C@H]1OC(O)[C@@H](O)[C@@H](O)[C@H]1O] | 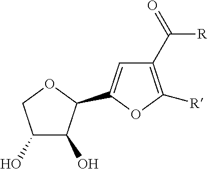 (the cyclized product is obtained predominantly) | 97% |
| 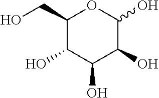 [SMILES: OC[C@H]1OC(O)[C@@H](O)[C@@H](O)[C@@H]1O] | 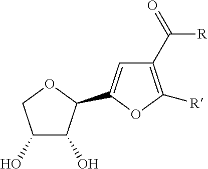 (the cyclized product is obtained predominantly) | 91% |

Standard Procedure:

The biosourced catalyst used in this reaction is an extract of Sedum plumbizincicola. It is of the CAT 4 type.

The weight of catalyst used in the reaction is adjusted as a function of the catalyst's metal content, so that the reaction uses 10% (in mol of limiting reagent) of the specific metal species of the catalyst selected.

The following are introduced into a 25-mL flask, equipped with a magnetic bar and a condenser: 2 mL of water/ethanol mixture (25/75), 1000 mg (5.55 mmol) of D-glucose, 515 mg (3.95 mmol) of ethyl acetoacetate and the weight of Lewis acid catalyst selected from the species mentioned above, so that the reaction uses 10% (in mol of limiting reagent) of the specific metal species of the catalyst selected. The mixture is heated at 80° C. for 24 h, and then evaporated to dryness under reduced pressure. The resultant solid is taken up in hot ethyl acetate (3×30 mL), the fractions are combined, concentrated, then the expected product is obtained by crystallization from this organic phase. Furan with a linear polyhydroxyl chain is obtained with a yield of 60%.

### Example 5

### Aldolization-Annelation-Diels-Alder Reaction Cascade

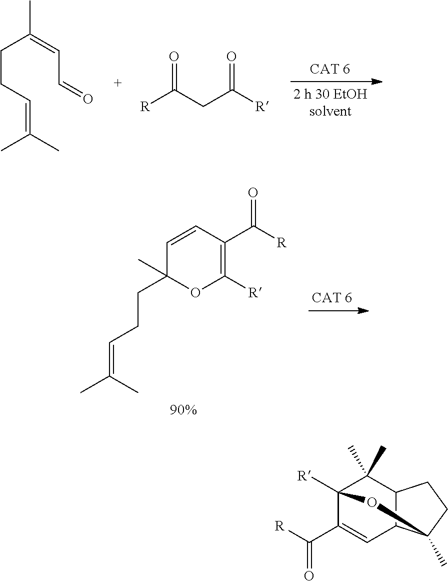

R, R′: alkyl or ester

To the best of our knowledge, this reaction is described for the first time with ethanol as solvent, as the prior references report the use of anhydrous solvents such as dichloromethane. Our catalyst therefore allows this reaction cascade to be carried out under conditions that are less harsh and more compatible with the principles of sustainable chemistry.

The polycyclic products resulting from this reaction comprise the skeleton of several natural compounds known for their antimalarial activity (pinnatal, isopinnatal, sterekunthal B).

### Example 6

### Reaction Cascade Involving Electrocyclization Reactions

Synthesis of a molecule having the characteristic skeleton of natural products that have shown antimalarial activity (pinnatal, sterekunthal and analogues, in particular present in the plant Kigelia pinnata, Bignoniaceae) was carried out by a one-pot synthesis process, involving a biosourced Lewis acid catalyst derived from Sedum.

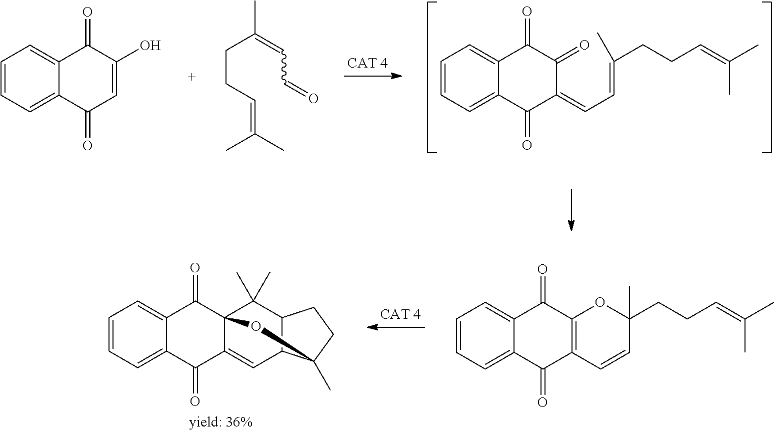

Procedure:
The biosourced catalyst used in this reaction will be selected from the catalysts derived from Sedum plumbizincicola. The weight of catalyst used in the reaction is adjusted as a function of the catalyst's metal content, so that the reaction uses 10% (in mol of limiting reagent) of the specific metal species of the catalyst selected.
A 10-mL flask, equipped with a magnetic bar and a condenser, is charged with 8 mL of anhydrous absolute ethanol and the weight of Lewis acid catalyst selected from the species mentioned above, so that the reaction uses 10% (in mol of limiting reagent) of the specific metal species of the catalyst selected. 152 mg (1.0 mmol) of citral is then added, with stirring at RT, followed by addition of 174 mg (1.0 mmol) of 2-hydroxy-1,4-naphthoquinone. The mixture is then taken to reflux for 5 hours, and then injected in GC-MS to monitor the progress. The characteristic ions m/z=308 and 293 are detected. The end product is purified by silica gel chromatography (hexane/ethyl acetate: 8/2 then 6/4). A red oil (Rf=0.5 in hexane/EtOAc 8/2) is obtained with a non-optimized yield of 36% for the whole synthesis.

Synthesis of octahydroacridines was carried out with microwave activation, using biosourced Lewis acid catalysts. These molecules have been described as inhibitors of gastric acid secretions. The reaction is complete in 3 minutes of activation, without solvent, on silica.

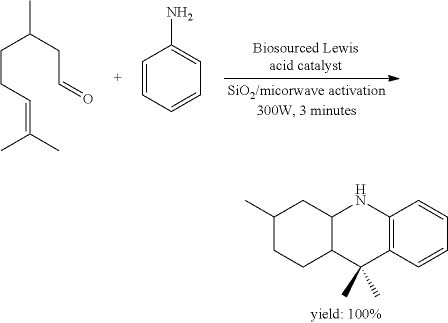

Procedure:
The biosourced catalyst used in this reaction will be selected from the catalysts derived from Sedum plumbizincicola. The weight of catalyst used in the reaction is adjusted as a function of the catalyst's metal content, so that the reaction uses 10% (in mol of limiting reagent) of the specific metal species of the catalyst selected.
Load a 20-mL scintillation vial with 540 mg of silica (35-70 μm) and the weight of Lewis acid catalyst selected from the species mentioned above, so that the reaction uses 10% (in mol of limiting reagent) of the specific metal species of the catalyst selected. Add 93 mg (1.0 mmol) of aniline and then 154 mg (1.0 mmol) of citronellal. Mix the paste obtained with a spatula for 1 minute. Irradiate in a microwave oven at 300 W, 3×1 minute (placing the vial in a sand bath, to absorb the excess radiation). A green powder is obtained. The latter is rinsed with dichloromethane, which remains colourless. The solution is analysed by GC-MS and reveals the presence of a single peak comprising the molecular ion m/z=229, characteristic of the expected product. Analysis in the presence of an internal standard (dodecane) allows a yield of 100% to be determined. Infrared analysis confirms complete formation of the product, with observation of bands at 3400 and 1605 cm⁻¹.

### Example 7

### Complete Synthesis of Perfume Molecules Using the Biosourced Lewis Acid Catalysts

The following complete syntheses were carried out using the biosourced catalysts derived from Sedum plumbizincicola. The weight of catalyst used in the reaction is adjusted as a function of the catalyst's metal content, so that the reaction uses 10% (in mol of limiting reagent) of the specific metal species of the catalyst selected.

Jasmacyclene:
The Lewis acid catalysts lead efficiently to the formation of esters by electrophilic addition of a carboxylic acid on a C═C double bond.

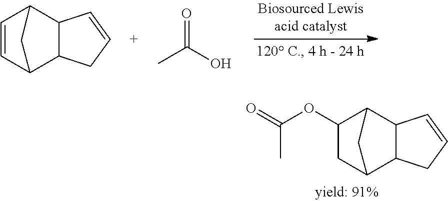

Procedure:
The biosourced catalyst used in this reaction will be selected from the catalysts derived from Sedum plumbizincicola. The weight of catalyst used in the reaction is adjusted as a function of the catalyst's metal content, so that the reaction uses 10% (in mol of limiting reagent) of the specific metal species of the catalyst selected.
The following are introduced into a sealed tube: 67 μL (0.5 mmol) of dicyclopentadiene (molten at 40° C.), 572 μL (10 mmol) of pure acetic acid and the weight of Lewis acid catalyst selected from the species mentioned above, so that the reaction uses 10% (in mol of limiting reagent) of the specific metal species of the catalyst selected. The tube is closed and heated in an oil bath at 120° C., for 4 to 24 hours.
For a heating time of 24 hours, Jasmacyclene (ester) is obtained with a yield of 91%. The reaction was carried out in the same way with propanoic and butyric acids, with similar yields. Similarly, the ethyl, propyl and butyl esters of norbornene were produced by the same methodology, with yields ranging from 92 to 100% in 24 hours.
   Stetter reaction: an aldolization reaction allowing access to a 1,4-dione by a green route:

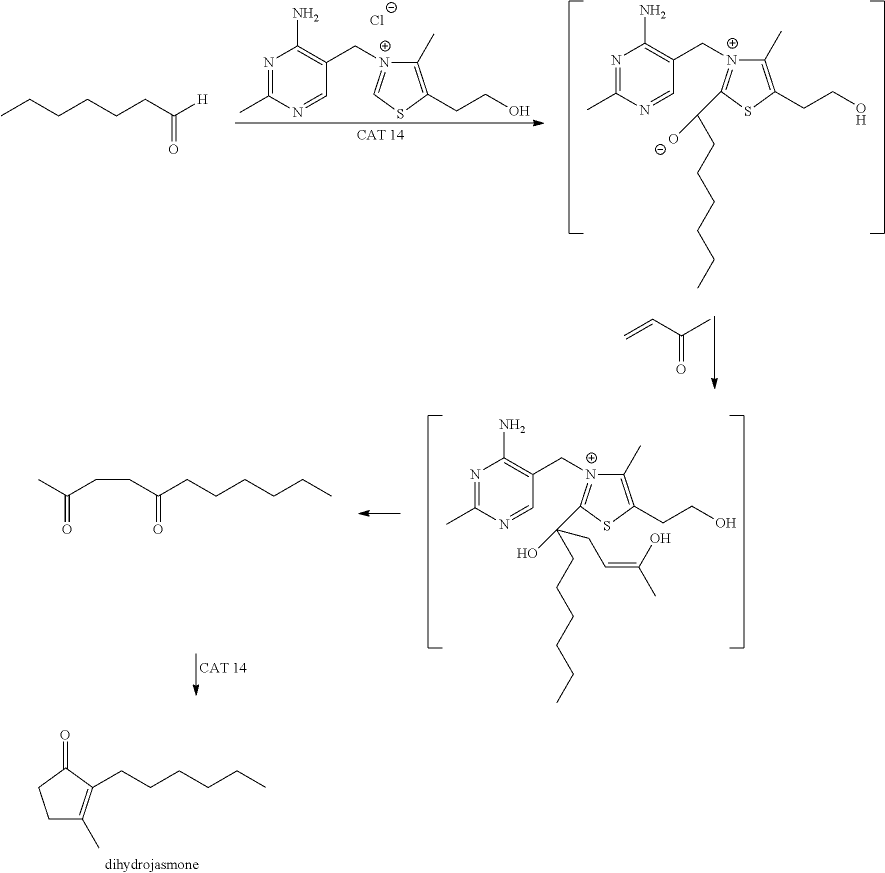

This reaction uses a natural substrate, thiamine. It constitutes the first step of a molecule useful in cosmetics, dihydrojasmone, according to a completely natural approach.
Operating conditions: 0.1 mmol of thiamine hydrochloride is introduced into 5 mL of acetonitrile. 2 mmol of 3-buten-2-one, then 100 mg of CAT 14 and 1 mmol of heptanal are added to the solution.
The reaction mixture is heated at 80° C. for 16 h and monitored by GC MS. The reaction is stopped after complete consumption of the heptanal. The dione is obtained with a high degree of purity and formation of the by-products of the conventional reaction using Et₃N instead of CAT14 is avoided (less than 1% of hydroxyketone and of enone).
At this stage of the reaction, 100 mg of CAT 14 is added again, heating is maintained at 80° C. until the intermediate 1,4-dione is consumed. Dihydrojasmone is obtained at an overall yield of 38% after filtration and evaporation.

The same process may be applied to the synthesis of hedione:

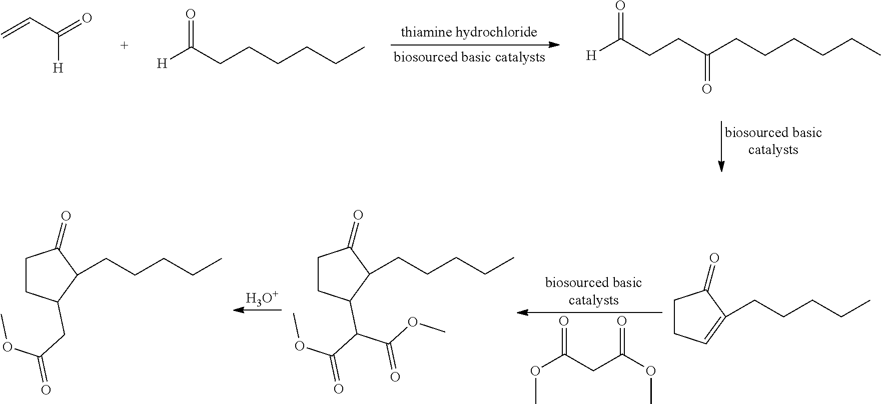

The process may also be generalized to the synthesis of cyclopentenones where the double bond is exocyclic:

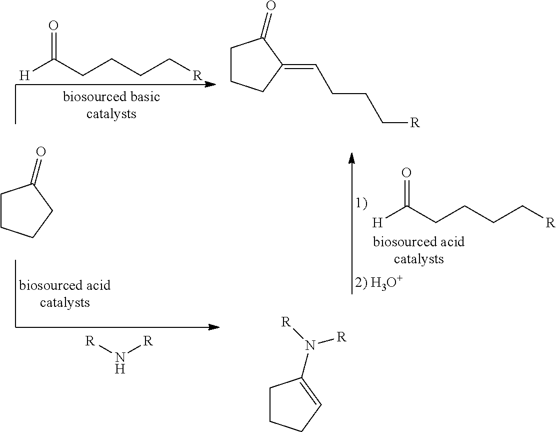

A successive sequence of catalysts with acid and basic properties can be exploited for the synthesis of campholenic aldehyde and its derivatives:

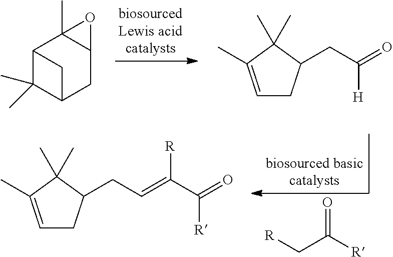

Synthesis of Isobutavan:
   Isobutavan may be prepared from vanillin by reaction with isobutyl chloride or isobutyric anhydride using the CT4, CAT6 and CAT 7 catalytic systems:

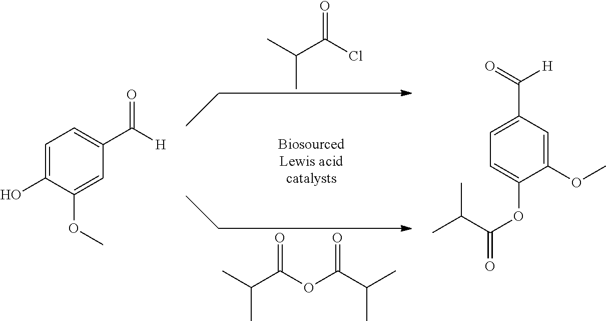

1. A composition comprising at least one metal catalyst, the metal of which has been accumulated after thermal treatment of a plant or part of a plant of the genus Sedum or of plants selected from Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, wherein the metal that has accumulated is at least one metal selected in particular from zinc (Zn), iron (Fe) or copper (Cu), said composition being substantially devoid of organic matter, for carrying out reactions of organic synthesis involving said catalyst.

2. The composition according to claim 1, wherein the plant or the part of a plant is of the genus Sedum or of the plant Potentilla griffithii.

3. The composition according to claim 1, wherein the plant or the part of a plant is selected from Sedum jinianum, Sedum plumbizincicola, Sedum alfredii and Potentilla griffithii, Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, in which said at least one metal is selected from zinc (Zn), calcium (Ca), magnesium (Mg), iron (Fe), cadmium (Cd) or copper (Cu), said composition optionally having been previously filtered and/or purified on resin and/or fixed on a support, after acid treatment.

4. The composition according to claim 3, wherein the acid treatment is carried out with hydrochloric acid, in particular gaseous HCl, 1N HCl to 12N HCl, sulphuric acid or trifluoromethanesulphonic acid.

5. The composition according to claim 1, wherein the plant or the part of a plant is selected from Sedum jinianum, Sedum plumbizincicola, Sedum alfredii, Potentilla griffithii, Arabis paniculata, Arabis gemmifera and Gentiana sp., wherein said at least one metal is selected from zinc (Zn), calcium (Ca), magnesium (Mg), iron (Fe), cadmium (Cd) or copper (Cu), said composition optionally having been previously filtered and/or purified on resin and/or fixed on a support, after hydration or basic treatment.

6. The composition according to claim 5, wherein the basic treatment is carried out by treating with a hydroxide, preferably sodium hydroxide or potassium hydroxide, until a pH of approximately 13 is obtained.

7. The composition according to claim 1, wherein the composition filtered on Celite or silica is optionally subsequently purified on ion-exchange resin.

8. The composition according to claim 1, wherein said plant is Sedum plumbizincicola (S. plumbizincicola).

9. The composition according to claim 1, wherein the Zn concentration is between approximately 4165 and approximately 45,000 mg/kg of dry weight of plant in the dried leaves of the plant S. plumbizincicola, is comprised between approximately 4100 and approximately 41000 mg/kg of dry weight of plant in the dried leaves of the plant S. jinianum, is comprised between approximately 4134 and approximately 5000 mg/kg of dry weight of plant in the dried leaves of the plant S. alfredii, is comprised between approximately 3870 and approximately 23,000 mg/kg of dry weight of plant in the dried leaves of the plant P. griffithii.

10. Process for the preparation of a composition substantially devoid of organic matter and comprising a metal catalyst consisting of one or more metals selected from zinc (Zn), calcium (Ca), magnesium (Mg), iron (Fe), cadmium (Cd) or copper (Cu), characterized in that it comprises the following steps:
a) dehydration of the biomass of a plant or of a plant extract of the genus Sedum or of the plant Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, that has accumulated at least one metal selected from zinc (Zn), calcium (Ca), magnesium (Mg), iron (Fe), cadmium (Cd) or copper (Cu),
b) grinding the dry biomass of a plant or of a plant extract obtained in step a),
c) thermal treatment of the ground mixture in a furnace preferably at a temperature below 500° C.
and if desired,
d) treatment of the ash obtained in step c) with an acid preferably selected from hydrochloric acid, nitric acid, sulphuric acid or trifluoromethanesulphonic acid followed if desired by dehydration of the solution obtained preferably at reduced pressure so as to obtain a dry residue
and solution obtained in step d) which, if desired, is subjected
e) to filtration preferably on Celite or on silica followed if desired by dehydration of the solution obtained preferably under reduced pressure so as to obtain a dry residue
and/or
f) to complete or partial purification on ion-exchange resins followed if desired by dehydration of the solution obtained preferably under reduced pressure so as to obtain a dry residue
and product in dry form obtained in step d), e) or f), which if desired
g) is mixed or treated in an acid medium with a support preferably selected from silica, montmorillonite, polygalacturonic acid, chitosan or a mixture of these products to obtain a supported catalyst.

11. A method of carrying out organic reactions, in particular the preparation of arylphosphonates and the Heck reaction, comprising adding to a reaction mixture a cocatalyst comprising a catalyst containing Ni(0) obtained from extracts of metallophyte plants that are hyperaccumulators of Ni(II) in combination with one of the compositions according to claim 1.

12. A method for carrying out the reactions of organic synthesis of functional transformations by Lewis acid catalysis selected from the:aromatic electrophilic substitution reactions such as Friedel-Crafts alkylating and acylating reactions and brominations, protection reactions such as the chemoselective tritylations of alcohols and amines, the acylations, in particular the acetylations of alcohols, phenols, thiols and amines, the silylations of alcohols, oximes, enolates, phenols, amines and anilines, the acetalizations, in particular of polyols or of sugars, formation of imines or amines, deprotection of functions in particular detritylation, concerted rearrangements such as the ene-reactions or the cycloadditions such as the Diels-Alder reaction, the pinacol or Beckmann rearrangement, the aldolization reactions such as the Claisen-Schmidt reaction, the Mukaiyama reaction or the reactions of the Knoevenagel type, dehydration or transfunctionalization reactions such as transamination or transtritylation reactions, the reactions for preparing polyheterocyclic structures such as porphyrinogens or dithienylpyrroles, the multicomponent reactions such as the triazole synthesis reactions, the Hantsch and Biginelli reactions, the syntheses of optionally substituted piperidines, of octahydroacridines, of chromenes, of pyridines and dihydropyridines, the syntheses of perfume molecules such as the cyclopentenones, Jasmacyclene, campholenic aldehyde, Isobutavan, the biomimetic reactions and hydride transfer reactions, the depolymerization reactions, the Garcia Gonzalez reaction, reaction cascades, redox reactions, comprising adding to a reaction mixture the composition containing at least one metal catalyst as described in claim 1.

13. A method for carrying out the reactions of organic synthesis comprising Lewis acid cocatalysis, preferably a hydrocyanation in combination with a catalyst of state (0) preferably obtained by reduction of a transition metal of state (II), preferably nickel, comprising adding to a reaction mixture the composition containing at least one metal catalyst as described in claim 1.

14. A method for carrying out reactions of organic synthesis involving said catalyst, the reactions being selected from the following reactions:
brominations, protection reactions such as the chemoselective tritylations of alcohols and amines, the acylations, in particular the acetylations of alcohols, phenols, thiols and amines, the silylations of alcohols, oximes, enolates, phenols, amines and anilines, formation of imines or amines, deprotection of functions in particular detritylation, concerted rearrangements such as the ene-reactions or cycloadditions, the pinacol or Beckmann rearrangement, Claisen-Schmidt reaction, Mukaiyama reaction or the reactions of the Knoevenagel type, the dehydration or transfunctionalization reactions such as the transamination or transtritylation reactions, the reactions for preparing polyheterocyclic structures such as porphyrinogens or dithienylpyrroles, the multicomponent reactions such as the triazole synthesis reactions, the Hantsch reactions, the syntheses of optionally substituted piperidines, the biomimetic reactions and hydride transfer reactions comprising adding to a reaction mixture a composition containing at least one metal catalyst, the metal of which is a metals derived selected from zinc (Zn), or copper (Cu) that has accumulated after thermal treatment of a plant or part of a plant, different from the genus Sedum or from the plant Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, said composition being substantially devoid of organic matter.

15. The method according to claim 14, wherein the composition containing at least one metal catalyst is used for carrying out the reactions of organic synthesis comprising Lewis acid cocatalysis, preferably a hydrocyanation in combination with a catalyst of state (0) preferably obtained by reduction of a transition metal of state (II), preferably nickel.

16. The method according to claim 13, wherein the catalyst obtained by reduction of nickel(II) is prepared by the action of a triarylphosphite, preferably triphenylphosphite or tritolylphosphite on an extract of a plant that is a hyperaccumulator of Ni(II), which is preferably Psychotria douarrei.

17. Composition obtained after thermal treatment of a plant or part of a plant of the genus Sedum or of the plant Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, substantially devoid of organic matter and in particular of chlorophyll containing at least one metal catalyst, the metal of which is selected in particular from Zn, or Cu, comprising at least one of said metals in the form of chloride or sulphate, and cellulosic degradation fragments such as cellobiose and/or glucose, and/or glucose degradation products such as 5-hydroxymethylfurfural and formic acid and less than approximately 2%, in particular less than approximately 0.2% by weight of C, in particular approximately 0.14%.

18. Composition as obtained by carrying out the process according to claim 10.

19. A method of preparing a composition according to claim 1, comprising
subjecting to a thermal treatment a plant or part of a plant of the genus Sedum or of plants selected from Potentilla griffithii, Arabis paniculata, Arabis gemmifera, Arabis alpina, Gentiana sp. Gentiana atuntsiensis, Silene viscidula, Corydalis davidii, Incarvillea deltoides, Corydalis pterygopetala, Picris divaricata, Sonchus asper, and
obtaining after said thermal treatment at least one metal that accumulated from said thermal treatment, the at least one metal selected from zinc (Zn), iron (Fe) or copper (Cu).
